(19) United States
(12) Patent Application Publication
(10) Pub. No.: US 2018/0297966 A1
(43) Pub. Date: Oct. 18, 2018

(54) COMPOUNDS, COMPOSITIONS, AND METHODS OF MAKING AND USING THE SAME

(71) Applicant: INNOVATIVE ANESTHETICS, LLC, Gainesville, FL (US)

(72) Inventors: Iryna O. Lebedyeva, Augusta, GA (US); Alexander A. Oliferenko, Gainesville, FL (US); John K. Neubert, Gainesville, FL (US); Gary I. Altschuler, Gainesville, FL (US); Robert A. Hromas, Gainesville, FL (US); David Ostrov, Gainesville, FL (US); William L. Castleman, Gainesville, FL (US)

(21) Appl. No.: 15/768,996

(22) PCT Filed: Oct. 17, 2016
(86) PCT No.: PCT/US16/57393

§ 371 Date: Apr. 17, 2018

Related U.S. Application Data
(60) Provisional application No. 62/243,012, filed on Oct. 17, 2015.

(51) Int. Cl. C07D 291/06; A61K 31/137; A61K 9/00; A61P 29/00; C07C 215/60; C07C 213/08; C07C 237/04; C07C 231/12; C07C 65/21; C07C 51/41; C07H 15/24; C07D 275/06; C07C 229/64; C07C 227/16; A61K 31/4458; A61K 31/445; A61K 31/167; A61K 31/381

(52) U.S. Cl. CPC C07D 291/06; A61K 31/381; A61K 9/0019; A61K 9/0014; A61P 29/00; C07C 215/60; C07C 213/08; C07C 237/04; C07C 231/12; C07C 65/21; C07C 51/41; C07H 15/24; C07D 275/06; C07C 229/64; C07C 227/16; A61K 31/4458; A61K 31/445; A61K 31/167; A61K 31/137

(57) ABSTRACT

Compounds are provided according to Formula (I), and hydrates thereof, and compositions thereof; and methods of using and making the same. Compounds of the present invention are contemplated useful for suppressing pain during cosmetic, medical, and dental procedures. In another aspect, provided herein is a composition comprising the compound of Formula (I) or hydrate thereof and a pharmaceutically acceptable carrier.

### CLAIM OF PRIORITY

This application claims priority from U.S. Ser. No. 62/243,012 filed Oct. 17, 2015, which is incorporated herein by reference in its entirety.

### BACKGROUND OF THE INVENTION

Local anesthesia is essential for suppressing pain during cosmetic, medical, or dental procedures such as, e.g., surgery, e.g., oral surgery. However, patients often perceive the receipt of intraoral local anesthesia as the most painful and sometimes the only objectionable part of these procedures and may therefore avoid obtaining necessary medical or dental care. For example, a significant number of patients detect an unpleasant bitter and metallic taste following intraoral injections of compositions comprising commonly used local anesthetics, e.g., lidocaine hydrochloride, which causes them to experience great anxiety during the medical or dental procedure. Further, hydrochloride salts that are commonly used in compositions formulated for local anesthesia, e.g., compositions comprising lidocaine hydrochloride and epinephrine hydrochloride, are acidic and can consequently cause additional pain and tissue damage. As a result, there exists a need for compositions comprising local anesthetics that possess a higher pH and/or do not possess objectionable tastes. Compounds, compositions, and methods of making and using the same are directed toward this end.

### SUMMARY OF THE INVENTION

The present invention includes compounds and compositions thereof and also contemplates their methods of making and use as local anesthetics in cosmetic, medical, and dental procedures.

In one aspect, the present invention provides a compound of Formula (I) or hydrate thereof:

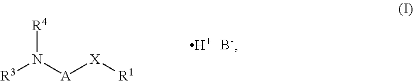

or hydrate thereof, wherein: B is a sweetener (e.g., saccharinate, acesulfamate, glycyrrherinate, mono-glycyrrhizinate, tri-glycyrrhizinate, vanillate, ferrulate, glycinate, cinnamate, enoxolone, cyclamate, steviol, aspartamate, di-glycyrrhinizinate, neotame); X is —C(O)O—, —OC(O)—, —C(O)NR^{A}—, or —NR^{A}C(O)—, or —CH(OR^{A})—, wherein R^{A} is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; R¹ is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; each of R³ and R⁴ is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, and heteroaryl is independently substituted with 0-5 occurrences of R^{Z};

A is:

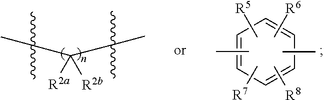

wherein: n is 1-5; each of R^{2a} and R^{2b} is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}, or if n is 1, R^{2a} or R^{2b} and R³ or R⁴, together with the atoms to which they are attached form a 3-8 membered ring independently substituted with 0-5 occurrences of R^{Z}; and each of R⁵, R⁶, R⁷, and R⁸ is independently hydrogen, alkyl (e.g., C₁-C₆ alkyl), C₁-C₆ alkoxy, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; and R^{Z} is halogen, C₁-C₆ alkyl, C₁-C₆ haloalkyl, C₁-C₆ heteroalkyl, —NHR^{Z1}, —NR^{Z1}R^{Z2}, —C(O)R^{Z1}, —C(O)R^{Z2}, —C(O)NR^{Z1}R^{Z2}, —NR^{Z1}C(O)R^{Z2}, —OR^{Z1}, —OR^{Z2}, cyano, or nitro, wherein R^{Z1} is hydrogen, C₁-C₆ alkyl, C₁-C₆ haloalkyl, C₁-C₆ heteroalkyl, hydroxyl, cyano, or nitro and R^{Z2} is carbocyclylalkyl, heterocycylalkyl, aralkyl or heteroaralkyl substituted with 0-5 occurrences of R^{Z1}; provided that the compound or hydrate thereof is not lidocaine saccharinate, lidocaine acesulfamate, bupivacaine saccharinate, bupivacaine acesulfamate, prilocaine saccharinate, prilocaine acesulfamate, procaine saccharinate, cinchocaine saccharinate, or benzocaine saccharinate.

In some embodiments, the compound is a compound of Formula (I-A):

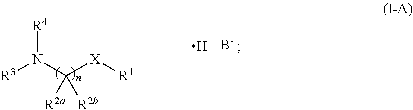

provided that the compound is not lidocaine saccharinate, lidocaine acesulfamate, bupivacaine saccharinate, bupivacaine acesulfamate, prilocaine saccharinate, prilocaine acesulfamate, procaine saccharinate, or cinchocaine saccharinate.

In some embodiments, the compound is a compound of Formula (I-B):

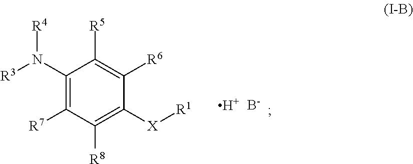

provided that the compound is not benzocaine saccharinate.

In some embodiments, B is saccharinate, acesulfamate, glycyrrherinate, mono-glycyrrhizinate, tri-glycyrrhizinate, vanillate, ferrulate, glycinate, cinnamate, enoxolone, cyclamate, steviol, aspartamate, di-glycyrrhinizinate, neotame

In some embodiments, the hydrate is a monohydrate. In some embodiments, X is —C(O)NR^{A}— or —NR^{A}C(O)—. In some embodiments, B is saccharinate or acesulfamate and R² and R³ are not —CH₂CH₃. In some embodiments, each of R^{2a} and R^{2b} is independently hydrogen. In some embodiments, R^{2a} and R^{2b} are hydrogen. In some embodiments, each of R^{2a} and R^{2b} is independently hydrogen or alkyl (e.g., C₁-C₆ alkyl) independently substituted with 0-5 occurrences of R^{Z} and n is 1. In some embodiments, each of R³ and R⁴ is independently hydrogen or alkyl (e.g., C₁-C₆ alkyl) independently substituted with 0-5 occurrences of R^{Z}. In some embodiments, each of R³ and R⁴ is independently hydrogen or —CH₃.

In some embodiments, if n is 1, R³ or R⁴ and R^{2a} or R^{2b}, together with the atoms to which they are attached form a 6-membered ring substituted with 0-5 occurrences of R^{Z}. In some embodiments, at least one of R⁵, R⁶, R⁷, and R⁸ is not hydrogen. In some embodiments, R³, R⁴, R⁵, R⁶, R⁷, and R⁸ are hydrogen. In some embodiments, R³ and R⁴ are hydrogen and X is —C(O)O—.

In some embodiments, the compound is a compound of Formula (I-C),

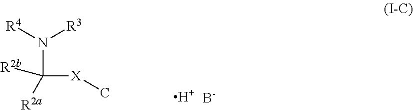

wherein C is a 5-10 membered ring substituted with 0-5 occurrences of R^{Z};

provided that the compound or hydrates thereof is not: lidocaine saccharinate, lidocaine acesulfamate, bupivacaine saccharinate, bupivacaine acesulfamate, cinchocaine saccharinate, prilocaine saccharinate, or prilocaine acesulfamate.

In some embodiments, C is,

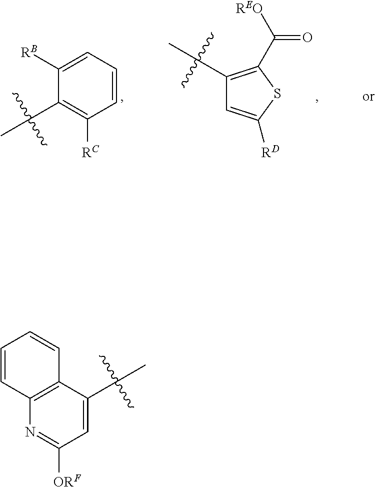

wherein each of R^{B}, R^{C}, R^{D}, R^{E}, or R^{F} is independently hydrogen or C₁-C₆ alkyl.

In some embodiments, R³ is n-butyl and R^{2a} and R⁴, together with the atoms to which they are attached form a 6-membered ring substituted with 0-5 occurrences of R^{Z}.

In some embodiments, R³ is methyl and R^{2a} and R⁴, together with the atoms to which they are attached form a 6-membered ring substituted with 0-5 occurrences of R^{Z}. In some embodiments, R^{2a} is methyl and R⁴ is n-propyl.

In some embodiments, the salt is represented by Formula (I-D):

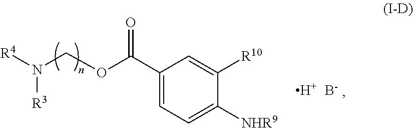

wherein R⁹ is hydrogen or C₁-C₆ alkyl and R¹⁰ is hydrogen or C₁-C₆ alkoxy;

provided that the compound is not procaine saccharinate.

In some embodiments, R³ and R⁴ are —CH₂CH₃. In some embodiments, n is 2. In some embodiments, R³ and R⁴ are —CH₂CH₃, R⁹ is hydrogen or C₁-C₆ alkyl, and R¹⁰ is hydrogen or C₁-C₆ alkoxy.

In some embodiments, the salt is represented by Formula (I-E):

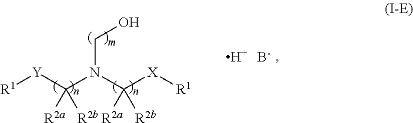

wherein m is 1, 2, 3, or 4 and Y is —NR^{A}C(O)— or —C(O)NR^{A}—.

In some embodiments, X is —C(O)NR^{A1}— and Y is —NR^{A1}C(O)—. In some embodiments, R¹ is aralkyl. In some embodiments, R^{2a} and R^{2b} are hydrogen. In some embodiments, R^{2a} and R^{2b} are hydrogen and R¹ is aralkyl. In some embodiments, n is 1 and m is 1.

In some embodiments, the compound is lidocaine glycyrrherinate, lidocaine mono-glycyrrhizinate, lidocaine tri-glycyrrhizinate, lidocaine vanillate, lidocaine ferrulate, lidocaine glycinate, lidocaine cinnamate, lidocaine enoxolone, lidocaine cyclamate, lidocaine steviol, lidocaine aspartamate, lidocaine di-glycyrrhinizinate, lidocaine neotame, tetracaine saccharinate, tetracaine acesulfamate, tetracaine glycyrrherinate, tetracaine mono-glycyrrhizinate, tetracaine tri-glycyrrhizinate, tetracaine vanillate, tetracaine ferrulate, tetracaine glycinate, tetracaine cinnamate, tetracaine enoxolone, tetracaine cyclamate, tetracaine steviol, tetracaine aspartamate, tetracaine di-glycyrrhinizinate, tetracaine neotame, bupivacaine glycyrrherinate, bupivacaine mono-glycyrrhizinate, bupivacaine tri-glycyrrhizinate, bupivacaine vanillate, bupivacaine ferrulate, bupivacaine glycinate, bupivacaine cinnamate, bupivacaine enoxolone, bupivacaine cyclamate, bupivacaine steviol, bupivacaine aspartamate, bupivacaine di-glycyrrhinizinate, bupivacaine neotame, mepivacaine saccharinate, mepivacaine acesulfamate, mepivacaine glycyrrherinate, mepivacaine mono-glycyrrhizinate, mepivacaine tri-glycyrrhizinate, mepivacaine vanillate, mepivacaine ferrulate, mepivacaine glycinate, mepivacaine cinnamate, mepivacaine enoxolone, mepivacaine cyclamate, mepivacaine steviol, mepivacaine aspartamate, mepivacaine di-glycyrrhinizinate, mepivacaine neotame, articaine saccharinate, articaine acesulfamate, articaine glycyrrherinate, articaine mono-glycyrrhizinate, articaine tri-glycyrrhizinate, articaine vanillate, articaine ferrulate, articaine glycinate, articaine cinnamate, articaine enoxolone, articaine cyclamate, articaine steviol, articaine aspartamate, articaine di-glycyrrhinizinate, articaine neotame, prilocaine glycyrrherinate, prilocaine mono-glycyrrhizinate, prilocaine tri-glycyrrhizinate, prilocaine vanillate, prilocaine ferrulate, prilocaine glycinate, prilocaine cinnamate, prilocaine enoxolone, prilocaine cyclamate, prilocaine steviol, prilocaine aspartamate, prilocaine di-glycyrrhinizinate, prilocaine neotame, procaine acesulfamate, procaine glycyrrherinate, procaine mono-glycyrrhizinate, procaine tri-glycyrrhizinate, procaine vanillate, procaine ferrulate, procaine glycinate, procaine cinnamate, procaine enoxolone, procaine cyclamate, procaine steviol, procaine aspartamate, procaine di-glycyrrhinizinate, procaine neotame, oxybuprocaine saccharinate, oxybuprocaine acesulfamate, oxybuprocaine glycyrrherinate, oxybuprocaine mono-glycyrrhizinate, oxybuprocaine tri-glycyrrhizinate, oxybuprocaine vanillate, oxybuprocaine ferrulate, oxybuprocaine glycinate, oxybuprocaine cinnamate, oxybuprocaine enoxolone, oxybuprocaine cyclamate, oxybuprocaine steviol, oxybuprocaine aspartamate, oxybuprocaine di-glycyrrhinizinate, oxybuprocaine neotame, ropivacaine saccharinate, ropivacaine acesulfamate, ropivacaine glycyrrherinate, ropivacaine mono-glycyrrhizinate, ropivacaine tri-glycyrrhizinate, ropivacaine vanillate, ropivacaine ferrulate, ropivacaine glycinate, ropivacaine cinnamate, ropivacaine enoxolone, ropivacaine cyclamate, ropivacaine steviol, ropivacaine aspartamate, ropivacaine di-glycyrrhinizinate, ropivacaine neotame, cinchocaine acesulfamate, cinchocaine glycyrrherinate, cinchocaine mono-glycyrrhizinate, cinchocaine tri-glycyrrhizinate, cinchocaine vanillate, cinchocaine ferrulate, cinchocaine glycinate, cinchocaine cinnamate, cinchocaine enoxolone, cinchocaine cyclamate, cinchocaine steviol, cinchocaine aspartamate, cinchocaine di-glycyrrhinizinate, cinchocaine neotame, benzocaine acesulfamate, benzocaine glycyrrherinate, benzocaine mono-glycyrrhizinate, benzocaine tri-glycyrrhizinate, benzocaine vanillate, benzocaine ferrulate, benzocaine glycinate, benzocaine cinnamate, benzocaine enoxolone, benzocaine cyclamate, benzocaine steviol, benzocaine aspartamate, benzocaine di-glycyrrhinizinate, benzocaine neotame, oxetacaine saccharinate, oxetacaine acesulfamate, oxetacaine glycyrrherinate, oxetacaine mono-glycyrrhizinate, oxetacaine tri-glycyrrhizinate, oxetacaine vanillate, oxetacaine ferrulate, oxetacaine glycinate, oxetacaine cinnamate, oxetacaine enoxolone, oxetacaine cyclamate, oxetacaine steviol, oxetacaine aspartamate, oxetacaine di-glycyrrhinizinate, or oxetacaine neotame. In some embodiments, the compound is epinephrine saccharinate, epinephrine acesulfamate, epinephrine glycyrrherinate, epinephrine mono-glycyrrhizinate, epinephrine tri-glycyrrhizinate, epinephrine vanillate, epinephrine ferrulate, epinephrine glycinate, epinephrine cinnamate, epinephrine enoxolone, epinephrine cyclamate, epinephrine steviol, epinephrine aspartamate, epinephrine di-glycyrrhinizinate, epinephrine neotame, levonordefrin saccharinate, levonordefrin acesulfamate, levonordefrin glycyrrherinate, levonordefrin mono-glycyrrhizinate, levonordefrin tri-glycyrrhizinate, levonordefrin vanillate, levonordefrin ferrulate, levonordefrin glycinate, levonordefrin cinnamate, levonordefrin enoxolone, levonordefrin cyclamate, levonordefrin steviol, levonordefrin aspartamate, levonordefrin di-glycyrrhinizinate, or levonordefrin neotame.

In another aspect, provided herein is a composition comprising the compound of Formula (I) or hydrate thereof and a pharmaceutically acceptable carrier.

In some embodiments, the composition further comprises epinephrine, levonodefrin, a salt of epinephrine, a salt of levonordefrin, or hydrate thereof. In some embodiments, the composition is formulated for injection. In some embodiments, the composition is formulated for oral, intraoral, subcutaneous, transdermal, or transmucosal administration. In some embodiments, the salt is epinephrine saccharinate, epinephrine acesulfamate, epinephrine glycyrrherinate, epinephrine mono-glycyrrhizinate, epinephrine tri-glycyrrhizinate, epinephrine vanillate, epinephrine ferrulate, epinephrine glycinate, epinephrine cinnamate, epinephrine enoxolone, epinephrine cyclamate, epinephrine steviol, epinephrine aspartamate, epinephrine di-glycyrrhinizinate, epinephrine neotame, levonordefrin saccharinate, levonordefrin acesulfamate, levonordefrin glycyrrherinate, levonordefrin mono-glycyrrhizinate, levonordefrin tri-glycyrrhizinate, levonordefrin vanillate, levonordefrin ferrulate, levonordefrin glycinate, levonordefrin cinnamate, levonordefrin enoxolone, levonordefrin cyclamate, levonordefrin steviol, levonordefrin aspartamate, levonordefrin di-glycyrrhinizinate, or levonordefrin neotame. In some embodiments, the pH of the compound is at least 3.0. In some embodiments, the pH of the composition ranges from about 3.6 to about 5.5. In some embodiments, the composition further comprises at least 0.000001% by weight of epinephrine, levonordefrin, a salt of epinephrine, a salt of levonodefrin, or hydrate thereof. In some embodiments, the further composition comprises from about 0.000001% by weight to about 10% by weight of epinephrine, levonordefrin, a salt of epinephrine, a salt of levonodefrin, or hydrate thereof. In some embodiments, the composition further comprises about 0.00001% by weight of epinephrine, levonordefrin, a salt of ephineprhine, a salt of levonordefrin, or hydrate thereof. In some embodiments, the composition comprises at least 0.0001% by weight of a compound of Formula (I) or hydrate thereof. In some embodiments, the composition comprises from about 0.1% by weight to about 10% by weight of a compound of Formula (I) or hydrate thereof. In some embodiments, the composition, when formulated for injection, comprises from about 1% by weight to about 3% by weight of a compound of Formula (I) or hydrate thereof. In some embodiments, the composition, when formulated for injection, comprises about 1% by weight of a compound of Formula (I) or hydrate thereof. In some embodiments, the composition, when formulated for topical or transdermal administration, comprises from about 1% by weight to about 10% by weight of a compound of Formula (I) or hydrate thereof. In some embodiments, the composition, when formulated for topical or transdermal administration, comprises about 5% by weight of a compound of Formula (I) or hydrate thereof.

In another aspect, the present invention provides a method for making the compound of Formula (I) or hydrate thereof, the method comprising dissolving the compound of Formula (II):

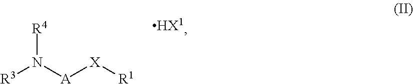

wherein:

X¹ is —OH or a halide anion (e.g., chloride, bromide, or iodide);

X is —C(O)O—, —OC(O)—, —C(O)NR^{A}—, or —NR^{A}C(O)—, or —CH(OR^{A})—, wherein R^{A} is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};

R¹ is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};

each of R³ and R⁴ is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, and heteroaryl is independently substituted with 0-5 occurrences of R^{Z};

A is:

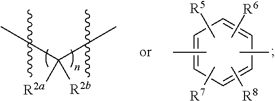

wherein:

n is 1-5;

each of R^{2a} and R^{2b} is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}, or

if n is 1, R^{2a} or R^{2b} and R³ or R⁴, together with the atoms to which they are attached form a 3-8 membered ring independently substituted with 0-5 occurrences of R^{Z}; and

each of R⁵, R⁶, R⁷, and R⁸ is independently hydrogen, alkyl (e.g., C₁-C₆ alkyl), C₁-C₆ alkoxy, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; and

R^{Z} is halogen, C₁-C₆ alkyl, C₁-C₆ haloalkyl, C₁-C₆ heteroalkyl, —NHR^{Z1}, —NR^{Z1}R^{Z2}, —C(O)R^{Z1}, —C(O)R^{Z2}, —C(O)NR^{Z1}R^{Z2}, —NR^{Z1}C(O)R^{Z2}, —OR^{Z1}, —OR^{Z2}, cyano, or nitro, wherein R^{Z1} is hydrogen, C₁-C₆ alkyl, C₁-C₆ haloalkyl, C₁-C₆ heteroalkyl, hydroxyl, cyano, or nitro and R^{Z2} is carbocyclylalkyl, heterocycylalkyl, aralkyl or heteroaralkyl substituted with 0-5 occurrences of R^{Z1};

provided that the following compounds or hydrates thereof are excluded:

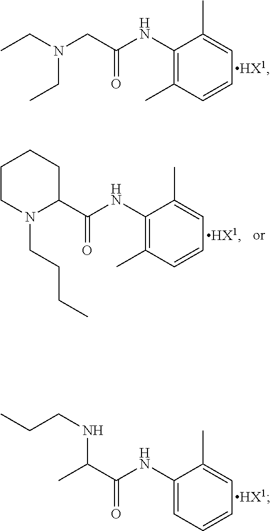 [SMILES: CCN(CC)CC(=O)Nc1c(C)cccc1C ; CCCCN1CCCCC1C(=O)Nc1c(C)cccc1C ; CCCNC(C)C(=O)Nc1ccccc1C]

and an organic salt in a solvent, wherein a metal halide precipitates or is solubilized and the compound of Formula (I) or hydrate thereof remains solubilized.

In some embodiments, the organic salt is sodium saccharinate, sodium acesulfamate, sodium glycyrrherinate, sodium mono-glycyrrhizinate, sodium tri-glycyrrhizinate, sodium vanillate, sodium ferrulate, sodium glycinate, sodium cinnamate, sodium enoxolone, sodium cyclamate, sodium steviol, sodium aspartamate, sodium di-glycyrrhinizinate, sodium neotame, potassium saccharinate, potassium acesulfamate, potassium glycyrrherinate, potassium mono-glycyrrhizinate, potassium tri-glycyrrhizinate, potassium vanillate, potassium ferrulate, potassium glycinate, potassium cinnamate, potassium enoxolone, potassium cyclamate, potassium steviol, potassium aspartamate, potassium di-glycyrrhinizinate, or potassium neotame. In some embodiments, X¹ is chloride or —OH. In some embodiments, the solvent is acetonitrile.

In another aspect, the present invention provides a method of suppressing pain experienced by a subject during a cosmetic, medical, or dental procedure, comprising administering to the subject an effective amount of a composition comprising a compound of Formula (I) or hydrate thereof:

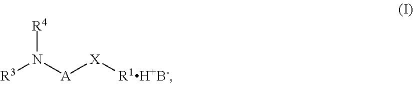

or hydrate thereof, wherein: B is a sweetener (e.g., saccharinate, acesulfamate, glycyrrherinate, mono-glycyrrhizinate, tri-glycyrrhizinate, vanillate, ferrulate, glycinate, cinnamate, enoxolone, cyclamate, steviol, aspartamate, di-glycyrrhinizinate, neotame); X is —C(O)O—, —OC(O)—, —C(O)NR^{A}—, or —NR^{A}C(O)—, or —CH(OR^{A})—, wherein R^{A} is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; R¹ is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; each of R³ and R⁴ is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, and heteroaryl is independently substituted with 0-5 occurrences of R^{Z}; A is:

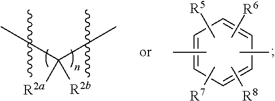

wherein: n is 1-5; each of R^{2a} and R^{2b} is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}, or if n is 1, R^{2a} or R^{2b} and R³ or R⁴, together with the atoms to which they are attached form a 3-8 membered ring independently substituted with 0-5 occurrences of R^{Z}; and each of R⁵, R⁶, R⁷, and R⁸ is independently hydrogen, alkyl (e.g., C₁-C₆ alkyl), C₁-C₆ alkoxy, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; and R^{Z} is halogen, C₁-C₆ alkyl, C₁-C₆ haloalkyl, C₁-C₆ heteroalkyl, —NHR^{Z1}, —NR^{Z1}R^{Z2}, —C(O)R^{Z1}, —C(O)R^{Z2}, —C(O)NR^{Z1}R^{Z2}, —NR^{Z1}C(O)R^{Z2}, —OR^{Z1}, —OR^{Z2}, cyano, or nitro, wherein R^{Z1} is hydrogen, C₁-C₆ alkyl, C₁-C₆ haloalkyl, C₁-C₆ heteroalkyl, hydroxyl, cyano, or nitro and R^{Z2} is carbocyclylalkyl, heterocycylalkyl, aralkyl or heteroaralkyl substituted with 0-5 occurrences of R^{Z1};

In some embodiments, the composition comprises lidocaine saccharinate, lidocaine acesulfamate, lidocaine glycyrrherinate, lidocaine mono-glycyrrhizinate, lidocaine tri-glycyrrhizinate, lidocaine vanillate, lidocaine ferrulate, lidocaine glycinate, lidocaine cinnamate, lidocaine enoxolone, lidocaine cyclamate, lidocaine steviol, lidocaine aspartamate, lidocaine di-glycyrrhinizinate, lidocaine neotame, tetracaine saccharinate, tetracaine acesulfamate, tetracaine glycyrrherinate, tetracaine mono-glycyrrhizinate, tetracaine tri-glycyrrhizinate, tetracaine vanillate, tetracaine ferrulate, tetracaine glycinate, tetracaine cinnamate, tetracaine enoxolone, tetracaine cyclamate, tetracaine steviol, tetracaine aspartamate, tetracaine di-glycyrrhinizinate, tetracaine neotame, bupivacaine saccharinate, bupivacaine acesulfamate, bupivacaine glycyrrherinate, bupivacaine mono-glycyrrhizinate, bupivacaine tri-glycyrrhizinate, bupivacaine vanillate, bupivacaine ferrulate, bupivacaine glycinate, bupivacaine cinnamate, bupivacaine enoxolone, bupivacaine cyclamate, bupivacaine steviol, bupivacaine aspartamate, bupivacaine di-glycyrrhinizinate, bupivacaine neotame, mepivacaine saccharinate, mepivacaine acesulfamate, mepivacaine glycyrrherinate, mepivacaine mono-glycyrrhizinate, mepivacaine tri-glycyrrhizinate, mepivacaine vanillate, mepivacaine ferrulate, mepivacaine glycinate, mepivacaine cinnamate, mepivacaine enoxolone, mepivacaine cyclamate, mepivacaine steviol, mepivacaine aspartamate, mepivacaine di-glycyrrhinizinate, mepivacaine neotame, articaine saccharinate, articaine acesulfamate, articaine glycyrrherinate, articaine mono-glycyrrhizinate, articaine tri-glycyrrhizinate, articaine vanillate, articaine ferrulate, articaine glycinate, articaine cinnamate, articaine enoxolone, articaine cyclamate, articaine steviol, articaine aspartamate, articaine di-glycyrrhinizinate, articaine neotame, prilocaine saccharinate, prilocaine acesulfamate, prilocaine glycyrrherinate, prilocaine mono-glycyrrhizinate, prilocaine tri-glycyrrhizinate, prilocaine vanillate, prilocaine ferrulate, prilocaine glycinate, prilocaine cinnamate, prilocaine enoxolone, prilocaine cyclamate, prilocaine steviol, prilocaine aspartamate, prilocaine di-glycyrrhinizinate, prilocaine neotame, procaine saccharinate, procaine acesulfamate, procaine glycyrrherinate, procaine mono-glycyrrhizinate, procaine tri-glycyrrhizinate, procaine vanillate, procaine ferrulate, procaine glycinate, procaine cinnamate, procaine enoxolone, procaine cyclamate, procaine steviol, procaine aspartamate, procaine di-glycyrrhinizinate, procaine neotame, oxybuprocaine saccharinate, oxybuprocaine acesulfamate, oxybuprocaine glycyrrherinate, oxybuprocaine mono-glycyrrhizinate, oxybuprocaine tri-glycyrrhizinate, oxybuprocaine vanillate, oxybuprocaine ferrulate, oxybuprocaine glycinate, oxybuprocaine cinnamate, oxybuprocaine enoxolone, oxybuprocaine cyclamate, oxybuprocaine steviol, oxybuprocaine aspartamate, oxybuprocaine di-glycyrrhinizinate, oxybuprocaine neotame, ropivacaine saccharinate, ropivacaine acesulfamate, ropivacaine glycyrrherinate, ropivacaine mono-glycyrrhizinate, ropivacaine tri-glycyrrhizinate, ropivacaine vanillate, ropivacaine ferrulate, ropivacaine glycinate, ropivacaine cinnamate, ropivacaine enoxolone, ropivacaine cyclamate, ropivacaine steviol, ropivacaine aspartamate, ropivacaine di-glycyrrhinizinate, ropivacaine neotame, cinchocaine saccharinate, cinchocaine acesulfamate, cinchocaine glycyrrherinate, cinchocaine mono-glycyrrhizinate, cinchocaine tri-glycyrrhizinate, cinchocaine vanillate, cinchocaine ferrulate, cinchocaine glycinate, cinchocaine cinnamate, cinchocaine enoxolone, cinchocaine cyclamate, cinchocaine steviol, cinchocaine aspartamate, cinchocaine di-glycyrrhinizinate, cinchocaine neotame, benzocaine saccharinate, benzocaine acesulfamate, benzocaine glycyrrherinate, benzocaine mono-glycyrrhizinate, benzocaine tri-glycyrrhizinate, benzocaine vanillate, benzocaine ferrulate, benzocaine glycinate, benzocaine cinnamate, benzocaine enoxolone, benzocaine cyclamate, benzocaine steviol, benzocaine aspartamate, benzocaine di-glycyrrhinizinate, benzocaine neotame, oxetacaine saccharinate, oxetacaine acesulfamate, oxetacaine glycyrrherinate, oxetacaine mono-glycyrrhizinate, oxetacaine tri-glycyrrhizinate, oxetacaine vanillate, oxetacaine ferrulate, oxetacaine glycinate, oxetacaine cinnamate, oxetacaine enoxolone, oxetacaine cyclamate, oxetacaine steviol, oxetacaine aspartamate, oxetacaine di-glycyrrhinizinate, or oxetacaine neotame. In some embodiments, the composition further comprises epinephrine, levonordefrin, a salt of epinephrine, a salt of levonodefrin, or hydrate thereof and a pharmaceutically acceptable carrier. In some embodiments, the salt of epinephrine is epinephrine saccharinate, epinephrine acesulfamate, epinephrine glycyrrherinate, epinephrine mono-glycyrrhizinate, epinephrine tri-glycyrrhizinate, epinephrine vanillate, epinephrine ferrulate, epinephrine glycinate, epinephrine cinnamate, epinephrine enoxolone, epinephrine cyclamate, epinephrine steviol, epinephrine aspartamate, epinephrine di-glycyrrhinizinate, or epinephrine neotame. In some embodiments, the salt of levonordefrin is levonordefrin saccharinate, levonordefrin acesulfamate, levonordefrin glycyrrherinate, levonordefrin mono-glycyrrhizinate, levonordefrin tri-glycyrrhizinate, levonordefrin vanillate, levonordefrin ferrulate, levonordefrin glycinate, levonordefrin cinnamate, levonordefrin enoxolone, levonordefrin cyclamate, levonordefrin steviol, levonordefrin aspartamate, levonordefrin di-glycyrrhinizinate, or levonordefrin neotame. In some embodiments, the composition is administered intraorally, epidurally, ocularly, intranasally, transdermally, subcutaneously, intramuscularly, or transmucosally. In some embodiments, the composition is formulated for injection. In some embodiments, the pH of the compound of Formula (I) or hydrate thereof is at least 3.0. In some embodiments, the pH of the compound of Formula (I) or hydrate thereof ranges from about 3.5 to about 5.5. In some embodiments, the pH of a composition comprising a compound of Formula (I) or hydrate thereof is at least 3.0. In some embodiments, the pH of a composition comprising a compound of Formula (I) or hydrate thereof ranges from about 3.6 to about 5.5. In some embodiments, the composition further comprises at least 0.000001% by weight of epinephrine, levonordefrin, a salt of epinephrine, a salt of levonodefrin, or hydrate thereof. In some embodiments, the composition further comprises from about 0.000001% by weight to about 10% by weight of epinephrine, levonordefrin, a salt of epinephrine, a salt of levonodefrin, or hydrate thereof. In some embodiments, the composition further comprises about 0.00001% by weight of epinephrine, levonordefrin, a salt of epinephrine, a salt of levonodefrin, or hydrate thereof. In some embodiments, the composition further comprises at least 0.0001% by weight of a compound of Formula (I) or hydrate thereof. In some embodiments, the composition further comprises from about 0.01% by weight to about 10% by weight of a compound of Formula (I) or hydrate thereof. In some embodiments, the composition, when formulated for injection, comprises from about 1% by weight to about 3% by weight of a compound of Formula (I) or hydrate thereof. In some embodiments, the composition, when formulated for injection, comprises about 1% by weight of a compound of Formula (I) or hydrate thereof. In some embodiments, the composition, when formulated for topical or transdermal administration, comprises from about 1% by weight to about 10% by weight of a compound of Formula (I) or hydrate thereof. In some embodiments, the composition, when formulated for topical or transdermal administration, comprises about 5% by weight of a compound of Formula (I) or hydrate thereof.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

Compounds described herein may also comprise one or more isotopic substitutions. For example, H may be in any isotopic form, including ¹H, ²H (D or deuterium), and ³H (T or tritium); C may be in any isotopic form, including ¹²C, 13C, and 14C; N may be in any isotopic form, including, ¹⁵N. O may be in any isotopic form, including ¹⁶O and ¹⁸O; and the like.

The compounds provided herein may also be represented in multiple tautomeric forms, in such instances, expressly includes all tautomeric forms of the compounds described herein, even though only a single tautomeric form may be represented (e.g., alkylation of a ring system may result in alkylation at multiple sites; all such reaction products are expressly included). All such isomeric forms of such compounds are expressly included.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. It should be further understood that the terms “groups” and “radicals” can be considered interchangeable when used herein. By way of example “an analogue” means one analogue or more than one analogue.

The term “halo” or “halogen” refers to any radical of fluorine, chlorine, bromine or iodine.

The term “alkyl” refers to a monovalent hydrocarbon chain that may be a straight chain or branched chain, containing the indicated number of carbon atoms. For example, C₁-C₁₂ alkyl indicates that the group may have from 1 to 12 (inclusive) carbon atoms in it. In certain aspects, the term “alkyl” refers to a monovalent hydrocarbon chain that may be a straight chain or branched chain, containing 1 to 6 carbon atoms. In other aspects, the term “alkyl” refers to a monovalent hydrocarbon chain that may be a straight chain or branched chain, containing 1 to 4 carbon atoms.

The term “haloalkyl” refers to an alkyl in which one or more hydrogen atoms are replaced by halo, and includes alkyl moieties in which all hydrogens have been replaced by halo (e.g., perfluoroalkyl).

“Alkoxy”, as used herein, refers to an alkyl group having an oxygen radical attached thereto. Representative alkoxyl groups include methoxy, ethoxy, propyloxy, tert-butoxy and the like.

The term “cyano” refers to a —CN radical.

The term “nitro” refers to an —NO₂ radical.

The term “aryl” refers to a monocyclic, bicyclic, or tricyclic aromatic hydrocarbon ring system. Examples of aryl moieties include, but are not limited to, phenyl, naphthyl, and anthracenyl.

The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. The term “carbocyclyl” refers to a non-aromatic, monocyclic, bicyclic, or tricyclic hydrocarbon ring system. Carbocyclyl groups include fully saturated ring systems (e.g., cycloalkyls), and partially saturated ring systems.

The term “cycloalkyl” as used herein includes saturated cyclic, bicyclic, tricyclic, or polycyclic hydrocarbon groups having 3 to 12 carbons. Any ring atom can be substituted (e.g., by one or more substituents). Examples of cycloalkyl moieties include, but are not limited to, cyclopropyl, cyclohexyl, methylcyclohexyl, adamantyl, and norbornyl.

The term “heteroaryl” refers to a fully aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms selected independently from N, O, or S if monocyclic, bicyclic, or tricyclic, respectively).

The term “heterocyclyl” refers to a nonaromatic, 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Examples of heterocyclyl include, but are not limited to, tetrahydrofuranyl, tetrahydropyranyl, piperidinyl, morpholino, pyrrolinyl, pyrimidinyl, and pyrrolidinyl.

Bicyclic and tricyclic ring systems containing one or more heteroatoms and both aromatic and non-aromatic rings are considered to be heterocyclyl groups according to the present definition. Such bicyclic or tricyclic ring systems may be alternately characterized as being an aryl or a heteroaryl fused to a carbocyclyl or heterocyclyl, particularly in those instances where the ring bound to the rest of the molecule is required to be aromatic. The terms “heteroarylalkyl” and “heteroaralkyl”, as used herein, refers to an alkyl group substituted with a heteroaryl group.

The term “carbocyclylalkyl”, as used herein, refers to an alkyl group substituted with a carbocyclyl group.

The term “heterocyclylalkyl”, as used herein, refers to an alkyl group substituted with a heterocyclyl group.

All ring systems (i.e, aryl, heteroaryl, carbocyclyl, cycloalkyl, heterocyclyl, etc.) or ring system portions of groups (e.g., the aryl portion of an aralkyl group) are optionally substituted at one or more substitutable carbon atoms with substituents including: halo, —C≡N, C₁-C₄ alkyl, ═O, C₃-C₇ carbocyle (e.g., cycloalkyl), C₁-C₄ alkyl, —OH, —O—(C₁-C₄ alkyl), —SH, —S—(C₁-C₄ alkyl), —(C₁-C₄ alkyl)-N(R^{b′})(R^{b′}), —N(R^{b′})(R^{b′}), —O—(C₁-C₄ alkyl)-N(R^{b′})(R^{b′}), —(C₁-C₄ alkyl)-O—(C₁-C₄ alkyl)-N(R^{b′})(R^{b′}), —C(O)—O(R^{b′}), —OC(O)(R^{b′}), —O—C(O)—O(R^{b′}), —C(O)—N(R^{b′})(R^{b′}), —N(R^{b′})—C(O)R^{b′}, —N(R^{b′})C(O)N(R^{b′})(R^{b′}), —N(R^{b′})—S(O)₁₋₂R^{b′}, —S(O)₁₋₂N(R^{b′})(R^{b′}), —N(R^{b′})S(O)₁₋₂N(R^{b′})(R^{b′}), —(C₁-C₄ alkyl)-C(O)—N(R^{b′})(R^{b′}), —O-(heteroaryl), —O-(heterocycle), —O-phenyl, -heteroaryl, -heterocycle, and -phenyl, wherein:

each R^{b′} is independently selected from hydrogen, —C₁-C₄ alkyl, carbocycle, carbocyclylalkyl, aryl, aralkyl, heteroaryl, heteroaralkyl, heterocyclyl, or heterocyclylalkyl; or two R^{b′} are taken together with the nitrogen atom to which they are bound to form a 4- to 8-membered saturated heterocycle optionally comprising one additional heteroatom selected from N, S, S(═O), S(═O)₂, and O,

any alkyl substituent is optionally further substituted with one or more of —OH, —O—(C₁-C₄ alkyl), halo, —NH₂, —NH(C₁-C₄ alkyl), or —N(C₁-C₄ alkyl)₂; and any carbon atom on a phenyl, carbocycle (e.g., cycloalkyl), heteroaryl or heterocycle substituent is optionally further substituted with one or more of —(C₁-C₄ alkyl), —(C₁-C₄ fluoroalkyl), —OH, —O—(C₁-C₄ alkyl), —O—(C₁-C₄ fluoroalkyl), halo, —NH₂, —NH(C₁-C₄ alkyl), or —N(C₁-C₄ alkyl)₂.

All heterocyclyl ring systems (and any heterocyclyl substituents on any ring system) are optionally substituted on one or more any substitutable nitrogen atom with —C₁-C₄ alkyl, oxo, fluoro-substituted C₁-C₄ alkyl, or acyl.

“Solvate” refers to forms of the compound that are associated with a solvent, usually by a solvolysis reaction. Forms of the compound that are associated with water is referred to as a “hydrate.” This physical association includes hydrogen bonding. Conventional solvents include water, ethanol, acetic acid, and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates.

Representative solvates include hydrates, ethanolates and methanolates.

The term “substituted” refers to the replacement of a hydrogen atom by another group.

These and other exemplary substituents are described in more detail in the Detailed Description, Examples, and claims. The invention is not intended to be limited in any manner by the above exemplary listing of substituents.

### Other Definitions

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates, cattle, pigs, horses, sheep, goats, rodents (e.g., Sprague Dawley® rats), cats, and/or dogs. In certain embodiments, the subject is a human. In certain embodiments, the subject is a non-human animal. The terms “human,” “patient,” and “subject” are used interchangeably herein.

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject.

A “withdrawal latency” or “withdrawal latency period” as used herein refers to the amount of time between when a subject, e.g., a rat, withdraws from a stimulus, e.g., a stimulus that produces pain, and receipt of said stimulus. In some embodiments, the stimulus is heat. In some embodiments, the withdrawal latency ranges from about 5 seconds to about 35 seconds.

### BRIEF DESCRIPTION OF THE DRAWINGS

FIG. 1 is an exemplary chart that depicts withdrawal latency of animals' hindpaws in response to a thermal stimulus following injection of a composition comprising a compound of Formula (I).

FIG. 2 is an exemplary chart that depicts the relative palatability of a compound of Formula (I) exhibited by animals.

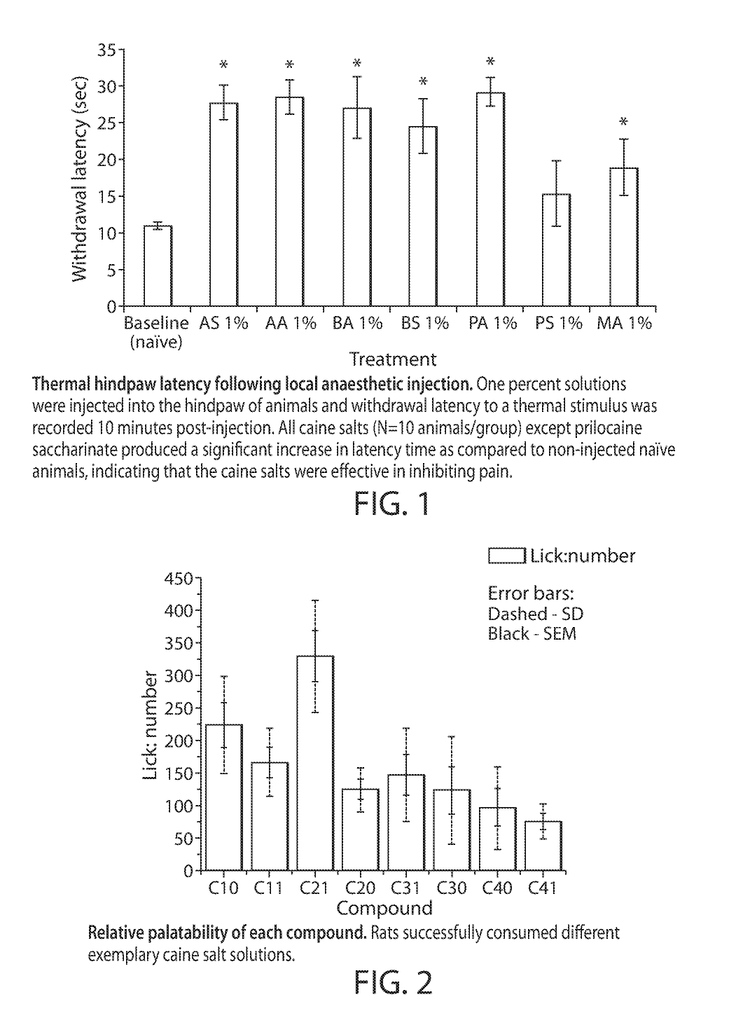

### DETAILED DESCRIPTION OF CERTAIN EMBODIMENTS OF THE INVENTION

As generally described herein, the present invention includes compounds and compositions thereof wherein the compounds are salts, wherein the anions of the salts are sweeteners, e.g., saccharinate, acesulfamate, glycyrrherinate, mono-glycyrrhizinate, tri-glycyrrhizinate, vanillate, ferrulate, glycinate, cinnamate, enoxolone, cyclamate, steviol, aspartamate, di-glycyrrhinizinate, neotame, and the cations are protonated organic amines. The chemical structures of the salts described herein are represented in such a way known to one skilled in the art. Saccharinate and acesulfamate anions may exist as their keto forms or enol forms as described below. In some embodiments, the compounds described herein can be formulated into compositions useful as local anesthetics for, e.g., surgery, e.g., oral surgery. In some embodiments, the compounds described herein are expected to be sweet-tasting and highly soluble in aqueous media, e.g., water or saline solution.

### Compounds

In one aspect, the present invention provides a compound of Formula (I) or hydrate thereof:

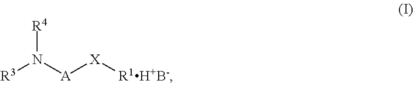

or hydrate thereof, wherein: B is a sweetener (e.g., saccharinate, acesulfamate, glycyrrherinate, mono-glycyrrhizinate, tri-glycyrrhizinate, vanillate, ferrulate, glycinate, cinnamate, enoxolone, cyclamate, steviol, aspartamate, di-glycyrrhinizinate, neotame); X is —C(O)O—, —OC(O)—, —C(O)NR^{A}—, or —NR^{A}C(O)—, or —CH(OR^{A})—, wherein R^{A} is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; R¹ is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; each of R³ and R⁴ is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, and heteroaryl is independently substituted with 0-5 occurrences of R^{Z};

A is:

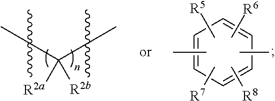

wherein: n is 1-5; each of R^{2a} and R^{2b} is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}, or if n is 1, R^{2a} or R^{2b} and R³ or R⁴, together with the atoms to which they are attached form a 3-8 membered ring independently substituted with 0-5 occurrences of R^{Z}; and each of R⁵, R⁶, R⁷, and R⁸ is independently hydrogen, alkyl (e.g., C₁-C₆ alkyl), C₁-C₆ alkoxy, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; and R^{Z} is halogen, C₁-C₆ alkyl, C₁-C₆ haloalkyl, C₁-C₆ heteroalkyl, —NHR^{Z1}, —NRR^{Z2}, —C(O)R^{Z1}, —C(O)R^{Z2}, —C(O)NR^{Z1}R^{Z2}, —NR^{Z1}C(O)R^{Z2}, —OR^{Z1}, —OR^{Z2}, cyano, or nitro, wherein R^{Z1} is hydrogen, C₁-C₆ alkyl, C₁-C₆ haloalkyl, C₁-C₆ heteroalkyl, hydroxyl, cyano, or nitro and R^{Z2} is carbocyclylalkyl, heterocycylalkyl, aralkyl or heteroaralkyl substituted with 0-5 occurrences of R^{Z1}; provided that the compound or hydrate thereof is not lidocaine saccharinate, lidocaine acesulfamate, bupivacaine saccharinate, bupivacaine acesulfamate, prilocaine saccharinate, prilocaine acesulfamate, procaine saccharinate, cinchocaine saccharinate, or benzocaine saccharinate.

In some embodiments, the compound is a compound of Formula (I-A):

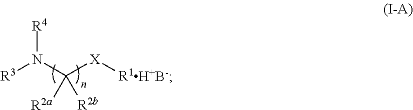

provided that the compound is not lidocaine saccharinate, lidocaine acesulfamate, bupivacaine saccharinate, bupivacaine acesulfamate, prilocaine saccharinate, prilocaine acesulfamate, procaine saccharinate, or cinchocaine saccharinate.

In some embodiments, the compound is a compound of Formula (I-B):

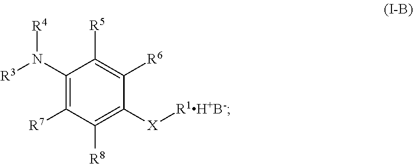

provided that the compound is not benzocaine saccharinate.

In some embodiments, B is saccharinate, acesulfamate, glycyrrherinate, mono-glycyrrhizinate, tri-glycyrrhizinate, vanillate, ferrulate, glycinate, cinnamate, enoxolone, cyclamate, steviol, aspartamate, di-glycyrrhinizinate, neotame

In some embodiments, the hydrate is a monohydrate. In some embodiments, X is —C(O)NR^{A}— or —NR^{A}C(O)—. In some embodiments, B is saccharinate or acesulfamate and R² and R³ are not —CH₂CH₃. In some embodiments, each of R^{2a} and R^{2b} is independently hydrogen. In some embodiments, R^{2a} and R^{2b} are hydrogen. In some embodiments, each of R^{2a} and R^{2b} is independently hydrogen or alkyl (e.g., C₁-C₆ alkyl) independently substituted with 0-5 occurrences of R^{Z} and n is 1. In some embodiments, each of R³ and R⁴ is independently hydrogen or alkyl (e.g., C₁-C₆ alkyl) independently substituted with 0-5 occurrences of R^{Z}. In some embodiments, each of R³ and R⁴ is independently hydrogen or —CH₃.

In some embodiments, if n is 1, R³ or R⁴ and R^{2a} or R^{2b}, together with the atoms to which they are attached form a 6-membered ring substituted with 0-5 occurrences of R^{Z}. In some embodiments, at least one of R⁵, R⁶, R⁷, and R is not hydrogen. In some embodiments, R³, R⁴, R⁵, R⁶, R⁷, and R⁸ are hydrogen. In some embodiments, R³ and R⁴ are hydrogen and X is —C(O)O—.

In some embodiments, the compound is a compound of Formula (I-C),

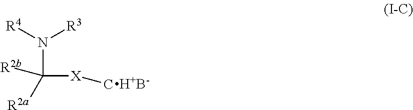

wherein C is a 5-10 membered ring substituted with 0-5 occurrences of R^{Z};

provided that the compound or hydrates thereof is not: lidocaine saccharinate, lidocaine acesulfamate, bupivacaine saccharinate, bupivacaine acesulfamate, cinchocaine saccharinate, prilocaine saccharinate, or prilocaine acesulfamate.

In some embodiments, C is,

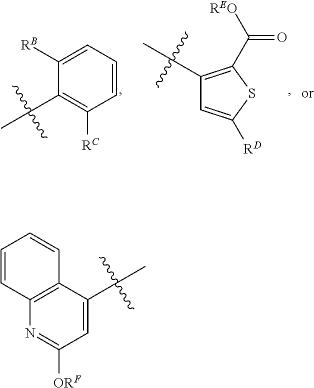

wherein each of R^{B}, R^{C}, R^{D}, R^{E}, or R^{F} is independently hydrogen or C₁-C₆ alkyl.

In some embodiments, R³ is n-butyl and R^{2a} and R⁴, together with the atoms to which they are attached form a 6-membered ring substituted with 0-5 occurrences of R^{Z}. In some embodiments, R³ is methyl and R^{2a} and R⁴, together with the atoms to which they are attached form a 6-membered ring substituted with 0-5 occurrences of R^{Z}. In some embodiments, R^{2a} is methyl and R⁴ is n-propyl.

In some embodiments, the salt is represented by Formula (I-D):

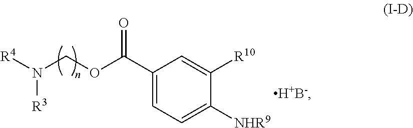

wherein R⁹ is hydrogen or C₁-C₆ alkyl and R¹⁰ is hydrogen or C₁-C₆ alkoxy;

provided that the compound is not procaine saccharinate.

In some embodiments, R³ and R⁴ are —CH₂CH₃. In some embodiments, n is 2. In some embodiments, R³ and R⁴ are —CH₂CH₃, R⁹ is hydrogen or C₁-C₆ alkyl, and R¹⁰ is hydrogen or C₁-C₆ alkoxy.

In some embodiments, the salt is represented by Formula (I-E):

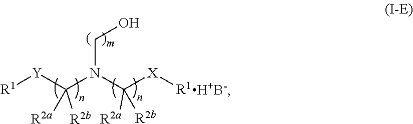

wherein m is 1, 2, 3, or 4 and Y is —NR^{A}C(O)— or —C(O)NR^{A}—.

In some embodiments, X is —C(O)NR^{A1}— and Y is —NR^{A1}C(O)—. In some embodiments, R¹ is aralkyl. In some embodiments, R^{2a} and R^{2b} are hydrogen. In some embodiments, R^{2a} and R^{2b} are hydrogen and R¹ is aralkyl. In some embodiments, n is 1 and m is 1.

In some embodiments, the compound is lidocaine glycyrrherinate, lidocaine mono-glycyrrhizinate, lidocaine tri-glycyrrhizinate, lidocaine vanillate, lidocaine ferrulate, lidocaine glycinate, lidocaine cinnamate, lidocaine enoxolone, lidocaine cyclamate, lidocaine steviol, lidocaine aspartamate, lidocaine di-glycyrrhinizinate, lidocaine neotame, tetracaine saccharinate, tetracaine acesulfamate, tetracaine glycyrrherinate, tetracaine mono-glycyrrhizinate, tetracaine tri-glycyrrhizinate, tetracaine vanillate, tetracaine ferrulate, tetracaine glycinate, tetracaine cinnamate, tetracaine enoxolone, tetracaine cyclamate, tetracaine steviol, tetracaine aspartamate, tetracaine di-glycyrrhinizinate, tetracaine neotame, bupivacaine glycyrrherinate, bupivacaine mono-glycyrrhizinate, bupivacaine tri-glycyrrhizinate, bupivacaine vanillate, bupivacaine ferrulate, bupivacaine glycinate, bupivacaine cinnamate, bupivacaine enoxolone, bupivacaine cyclamate, bupivacaine steviol, bupivacaine aspartamate, bupivacaine di-glycyrrhinizinate, bupivacaine neotame, mepivacaine saccharinate, mepivacaine acesulfamate, mepivacaine glycyrrherinate, mepivacaine mono-glycyrrhizinate, mepivacaine tri-glycyrrhizinate, mepivacaine vanillate, mepivacaine ferrulate, mepivacaine glycinate, mepivacaine cinnamate, mepivacaine enoxolone, mepivacaine cyclamate, mepivacaine steviol, mepivacaine aspartamate, mepivacaine di-glycyrrhinizinate, mepivacaine neotame, articaine saccharinate, articaine acesulfamate, articaine glycyrrherinate, articaine mono-glycyrrhizinate, articaine tri-glycyrrhizinate, articaine vanillate, articaine ferrulate, articaine glycinate, articaine cinnamate, articaine enoxolone, articaine cyclamate, articaine steviol, articaine aspartamate, articaine di-glycyrrhinizinate, articaine neotame, prilocaine glycyrrherinate, prilocaine mono-glycyrrhizinate, prilocaine tri-glycyrrhizinate, prilocaine vanillate, prilocaine ferrulate, prilocaine glycinate, prilocaine cinnamate, prilocaine enoxolone, prilocaine cyclamate, prilocaine steviol, prilocaine aspartamate, prilocaine di-glycyrrhinizinate, prilocaine neotame, procaine acesulfamate, procaine glycyrrherinate, procaine mono-glycyrrhizinate, procaine tri-glycyrrhizinate, procaine vanillate, procaine ferrulate, procaine glycinate, procaine cinnamate, procaine enoxolone, procaine cyclamate, procaine steviol, procaine aspartamate, procaine di-glycyrrhinizinate, procaine neotame, oxybuprocaine saccharinate, oxybuprocaine acesulfamate, oxybuprocaine glycyrrherinate, oxybuprocaine mono-glycyrrhizinate, oxybuprocaine tri-glycyrrhizinate, oxybuprocaine vanillate, oxybuprocaine ferrulate, oxybuprocaine glycinate, oxybuprocaine cinnamate, oxybuprocaine enoxolone, oxybuprocaine cyclamate, oxybuprocaine steviol, oxybuprocaine aspartamate, oxybuprocaine di-glycyrrhinizinate, oxybuprocaine neotame, ropivacaine saccharinate, ropivacaine acesulfamate, ropivacaine glycyrrherinate, ropivacaine mono-glycyrrhizinate, ropivacaine tri-glycyrrhizinate, ropivacaine vanillate, ropivacaine ferrulate, ropivacaine glycinate, ropivacaine cinnamate, ropivacaine enoxolone, ropivacaine cyclamate, ropivacaine steviol, ropivacaine aspartamate, ropivacaine di-glycyrrhinizinate, ropivacaine neotame, cinchocaine acesulfamate, cinchocaine glycyrrherinate, cinchocaine mono-glycyrrhizinate, cinchocaine tri-glycyrrhizinate, cinchocaine vanillate, cinchocaine ferrulate, cinchocaine glycinate, cinchocaine cinnamate, cinchocaine enoxolone, cinchocaine cyclamate, cinchocaine steviol, cinchocaine aspartamate, cinchocaine di-glycyrrhinizinate, cinchocaine neotame, benzocaine acesulfamate, benzocaine glycyrrherinate, benzocaine mono-glycyrrhizinate, benzocaine tri-glycyrrhizinate, benzocaine vanillate, benzocaine ferrulate, benzocaine glycinate, benzocaine cinnamate, benzocaine enoxolone, benzocaine cyclamate, benzocaine steviol, benzocaine aspartamate, benzocaine di-glycyrrhinizinate, benzocaine neotame, oxetacaine saccharinate, oxetacaine acesulfamate, oxetacaine glycyrrherinate, oxetacaine mono-glycyrrhizinate, oxetacaine tri-glycyrrhizinate, oxetacaine vanillate, oxetacaine ferrulate, oxetacaine glycinate, oxetacaine cinnamate, oxetacaine enoxolone, oxetacaine cyclamate, oxetacaine steviol, oxetacaine aspartamate, oxetacaine di-glycyrrhinizinate, or oxetacaine neotame. In some embodiments, the compound is epinephrine saccharinate, epinephrine acesulfamate, epinephrine glycyrrherinate, epinephrine mono-glycyrrhizinate, epinephrine tri-glycyrrhizinate, epinephrine vanillate, epinephrine ferrulate, epinephrine glycinate, epinephrine cinnamate, epinephrine enoxolone, epinephrine cyclamate, epinephrine steviol, epinephrine aspartamate, epinephrine di-glycyrrhinizinate, epinephrine neotame, levonordefrin saccharinate, levonordefrin acesulfamate, levonordefrin glycyrrherinate, levonordefrin mono-glycyrrhizinate, levonordefrin tri-glycyrrhizinate, levonordefrin vanillate, levonordefrin ferrulate, levonordefrin glycinate, levonordefrin cinnamate, levonordefrin enoxolone, levonordefrin cyclamate, levonordefrin steviol, levonordefrin aspartamate, levonordefrin di-glycyrrhinizinate, or levonordefrin neotame.

Compounds of the present invention, e.g., a compound of Formula (I) or hydrate thereof, can be organic salts in which the cation is a protonated member of the caine family. A general structure of a caine salt provided by the present invention can be generally represented as a compound of Formula (A-1):

Caine•H⁺B⁻ (A-1)

wherein anion B is a sweetener. Exemplary caines of Formula (A-1) are depicted in Table 1a and examples of B are shown in Table 1b below. The caines shown below are depicted in their neutral form, but are expected to be protonated as the component of a salt described herein, e.g., a compound of Formula (I).

TABLE 1a
Caine

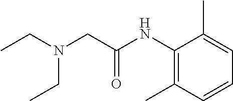 [SMILES: CCN(CC)CC(=O)Nc1c(C)cccc1C]

Lidocaine

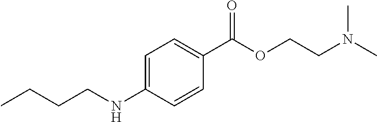 [SMILES: CCCCNc1ccc(C(=O)OCCN(C)C)cc1]

Tetracaine

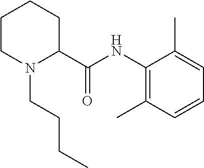 [SMILES: CCCCN1CCCCC1C(=O)Nc1c(C)cccc1C]

Bupivacaine

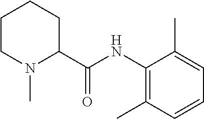 [SMILES: Cc1cccc(C)c1NC(=O)C1CCCCN1C]

Mepivacaine

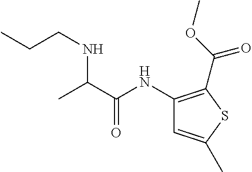 [SMILES: CCCNC(C)C(=O)Nc1cc(C)sc1C(=O)OC]

Articaine

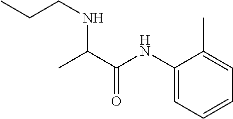 [SMILES: CCCNC(C)C(=O)Nc1ccccc1C]

Prilocaine

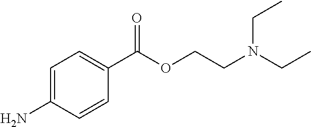 [SMILES: CCN(CC)CCOC(=O)c1ccc(N)cc1]

Procaine

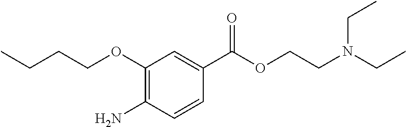 [SMILES: CCCCOc1cc(C(=O)OCCN(CC)CC)ccc1N]

Oxybuprocaine

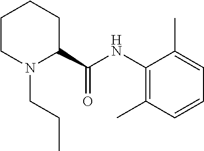

Ropivacaine

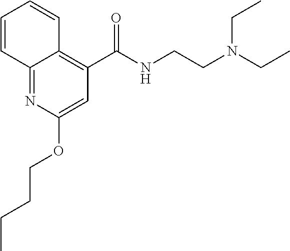 [SMILES: CCCCOc1cc(C(=O)NCCN(CC)CC)c2ccccc2n1]

Cinchocaine

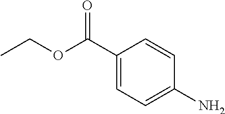

Benzocaine

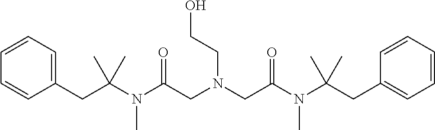 [SMILES: CN(C(=O)CN(CCO)CC(=O)N(C)C(C)(C)Cc1ccccc1)C(C)(C)Cc1ccccc1]

Oxetacaine

TABLE 1b
Sweetener (B)

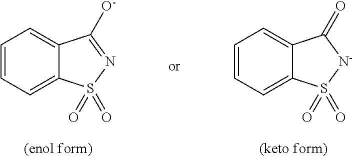 [SMILES: O=S1(=O)N=C([O-])c2ccccc21 ; O=C1[N-]S(=O)(=O)c2ccccc21]

Saccharinate

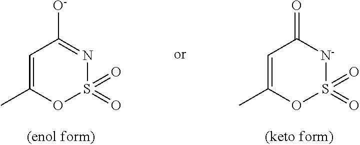

Acesulfamate

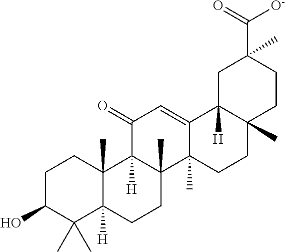

Enoxolone anion

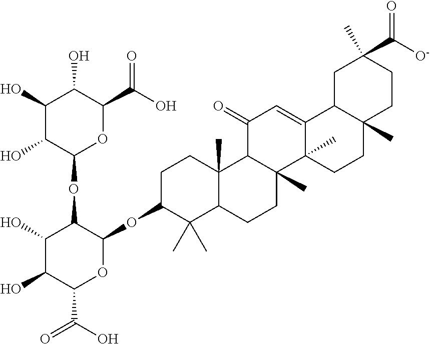

Mono-glycyrrhizinate

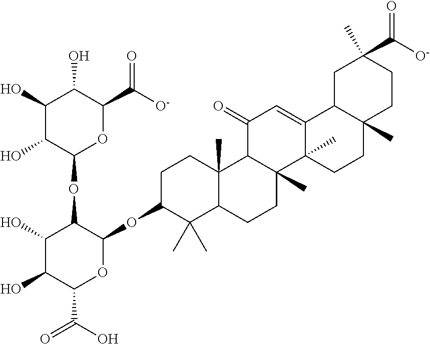

Di-glycyrrhizinate

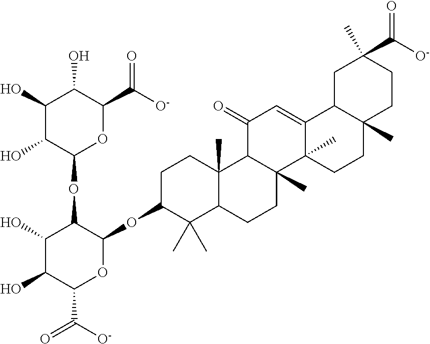

Tri-glycyrrhizinate

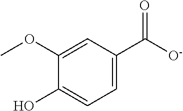

Vanillate

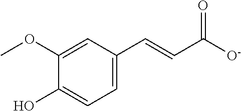

Ferrulate

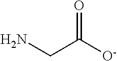

Glycinate

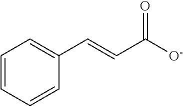 [SMILES: O=C([O-])/C=C/c1ccccc1]

Cinnamate

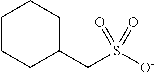 [SMILES: O=S(=O)([O-])CC1CCCCC1]

Cyclamate

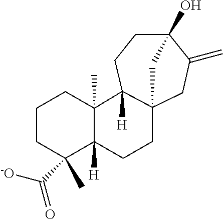

Steviol

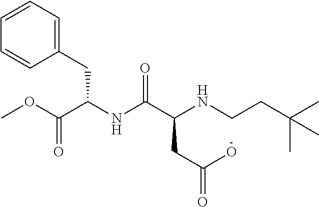 [SMILES: COC(=O)[C@H](Cc1ccccc1)NC(=O)[C@H](CC(=O)O)NCCC(C)(C)C]

Neotame

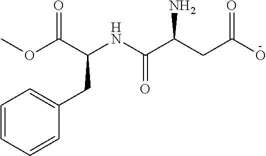 [SMILES: COC(=O)[C@H](Cc1ccccc1)NC(=O)[C@@H](N)CC(=O)O]

Aspartamate

Compounds of the present invention can also be other organic salts as depicted by Formula (A-2):

E•H⁺B⁻ (A-2)

wherein cationic component E can be an organic amine, e.g., epinephrine or levonordefrin as shown in Table 2a and anion B is a sweetener as shown in Table 2b. As with the caines of Table 1a, the species shown in Table 2a are depicted in their neutral form, but are expected to be protonated as the component of a salt described herein, e.g., a compound of Formula (I).

TABLE 2a
Component E

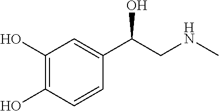

Epinephrine

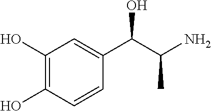

Levonordefrin

TABLE 2b
Sweetener (B)

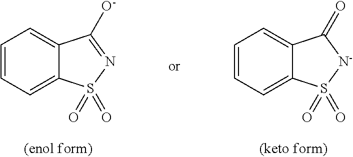 [SMILES: O=S1(=O)N=C([O-])c2ccccc21 ; O=C1[N-]S(=O)(=O)c2ccccc21]

Saccharinate

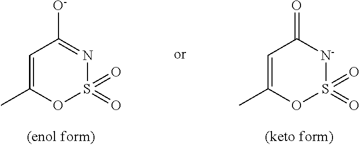

Acesulfamate

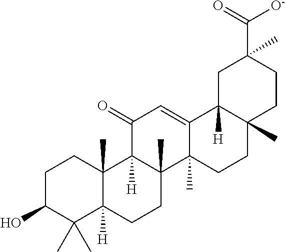

Enoxolone

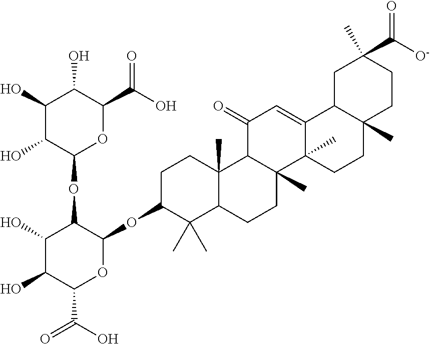

Mono-glycyrrhizinate

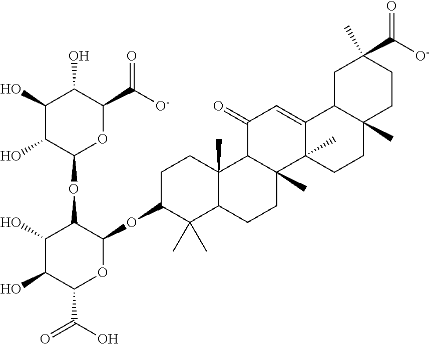

Di-glycyrrhizinate

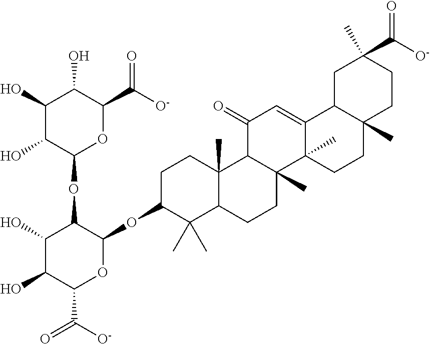

Tri-glycyrrhizinate

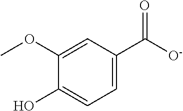

Vanillate

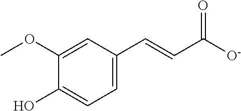

Ferrulate

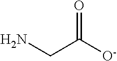

Glycinate

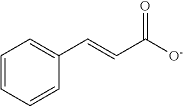 [SMILES: O=C([O-])/C=C/c1ccccc1]

Cinnamate

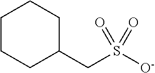 [SMILES: O=S(=O)([O-])CC1CCCCC1]

Cyclamate

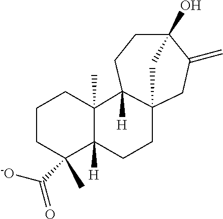

Steviol

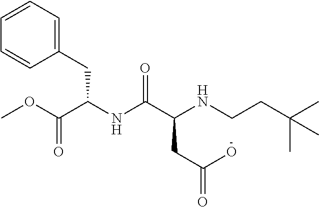 [SMILES: COC(=O)[C@H](Cc1ccccc1)NC(=O)[C@H](CC(=O)O)NCCC(C)(C)C]

Neotame

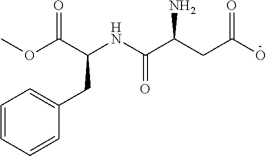 [SMILES: COC(=O)[C@H](Cc1ccccc1)NC(=O)[C@@H](N)CC(=O)O]

Aspartamate

Salts in which the cation is a protonated caine species and the anion is a sweetener can be named as the combination of the name of the caine species followed by the name of the anionic sweetener. For example, a salt in which the cation is protonated oxybuprocaine and the anion is saccharinate can be named as oxybuprocaine saccharinate. Similarly, compounds of Formula (A-2), e.g., a salt in which the cation is protonated epinephrine and the anion is acesulfamate, can be identified as epinephrine acesulfamate and a salt in which the cation is protonated levonodefrin and the anion is vanillate can be named as levonodefrin vanillate.

### Compositions, Formulations, and Routes of Administration

In another aspect, provided herein are compositions comprising a compound described herein, e.g., a compound of Formula (I), and a pharmaceutically acceptable carrier. In some embodiments, with respect to the composition, the pharmaceutically acceptable carrier is an injectable carrier, an oral carrier, or a topical carrier. In some embodiments, a composition can comprise at least 0.0001%, e.g., from about 0.01% by weight to about 10% by weight, of a compound of Formula (I) or hydrate thereof. In some embodiments, a composition can comprise about 1% by weight of a compound of Formula (I) or hydrate thereof.

### Formulations and Routes of Administration

The present invention describes herein pharmaceutically acceptable formulations of compounds described herein, e.g., compounds of Formula (I) or hydrates thereof. The compositions provided herein can be administered by a variety of routes including oral, intraoral, transdermal, subcutaneous, intravenous, intramuscular, intranasal, and transmucosal administrations. Depending on the intended route of delivery, the compounds provided herein, e.g., compounds of Formula (I) or hydrates thereof, can be formulated as, e.g., injectable compositions, oral compositions, sprayable compositions, tablets, capsules, foams, or gels, or ointments, lotions, or patches that can be topically administered.

Generally, the compounds provided herein, e.g., a compound of Formula (I) are administered in an effective amount generally ranging from about 0.0001% by weight to about 10% by weight, e.g., from about 0.3% by weight to about 5% by weight, of the total composition. In some embodiments, a compound provided herein, e.g., a compound of Formula (I) or hydrate thereof, is administered in an amount of about 1% by weight of the total composition.

Compositions for administration can take the form of bulk liquid solutions or suspensions, or bulk powders. The compositions can be presented in unit dosage forms to facilitate accurate dosing. The term “unit dosage forms” refers to physically discrete units suitable as unitary dosages for human subjects and other mammals, each unit containing a predetermined quantity of active material calculated to produce the desired effect, in association with a suitable pharmaceutical excipient. Typical unit dosage forms include prefilled, premeasured ampules or syringes of the liquid compositions or pills, tablets, capsules or the like in the case of solid compositions. In such compositions, the compound, e.g., a compound of Formula (I) or hydrate thereof, is usually a minor component (e.g., from about 0.0001% by weight to about 10% by weight, e.g., from about 0.3% by weight to about 5% by weight) with the remainder being various vehicles or carriers and processing aids helpful for forming the desired dosing form. In some embodiments, compositions can be formulated for use in eye drops or for spraying into, e.g., nostrils or the mouth using pharmaceutically acceptable carriers and excipients known in the art.

Other liquid forms suitable for administration can include a suitable aqueous or nonaqueous vehicle with buffers, suspending and dispensing agents, colorants, and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; or a glidant such as colloidal silicon dioxide.

The compositions and components described herein can be provided in the form of an oral rinse. Ingredients of such an oral rinse typically include one or more of an active ingredient (e.g., a compound of Formula (I) or hydrate thereof, e.g., from at least 0.0008%, from at least 0.001%, at least 0.003%, at least 0.004%, from about 0.001% to about 0.8%, from about 0.001% to about 0.005%, from about 0.003% to about 0.8%, from about 0.003% to about 0.02%, from about 0.003% to about 0.01%, from about 0.004% to about 0.8%, from about 0.004% to about 0.02%, from about 0.004% to about 0.01%), a non-fermentable sugar (e.g., from about 1% to about 70%, about 5% to about 70%, about 10% to about 70%, about 17% to about 70%, about 1% to about 65%, about 5% to about 70%, about 10% to about 70%, about 17% to about 65%, about 22% to about 33%), a thickener (e.g., from about 1% to about 20%, about 5% to about 15%, about 10% to about 15%, about 12.5%), a surfactant (e.g., from about 0.1% to about 2%, about 0.5% to about 2.5%, about 1% to about 2%, about 1%), and a preservative (e.g., from about 0.01% to about 4%, from about 0.01 to about 0.4%, from about 0.01 to about 0.2%, from about 0.2% to about 0.4%, about 0.1%). Such oral rinses may optionally include one or more of an anti-caries agent (from about 0% to about 0.1% as fluoride ion), an anti-calculus agent (from about 0.1% to about 3%), an antiseptic agent (e.g., thymol), an anesthetic agent (e.g., a local anesthetic agent (e.g., menthol)), a cleaning agent (e.g., methyl salicylate), a whitening agent (e.g., hydrogen peroxide), a base (e.g., sodium hydroxide), and a desensitizing agent (e.g., potassium nitrate).

In some embodiments, injectable formulations can be administered intraorally, instramuscularly, subcutaneously, or transmucosally. In some embodiments, injections are administered in the nose. Injectable compositions are typically based upon aqueous-based carriers, e.g., injectable sterile saline or phosphate-buffered saline or other injectable carriers known in the art. The active compound, e.g., a compound of Formula (I) or hydrate thereof, in such compositions is typically a minor component, often being from about 0.3% by weight to about 5% by weight and preferably from about 1% by weight to about 3% by weight, with the remainder being the injectable carrier and the like. In some embodiments, the active compound in such compositions is about 1% by weight with the remainder being the injectable carrier and the like.

Transdermal compositions are typically formulated as a topical ointment or cream containing the active ingredient(s), e.g., a compound of Formula (I) or hydrate thereof, generally in an amount ranging from about 0.1% by weight to about 10% by weight, e.g., from about 0.5% by weight to about 10% by weight, of the total composition. In some embodiments, the transdermal composition comprises about 5% by weight of the total composition. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable, or topically administrable compositions are merely representative. Other materials as well as processing techniques and the like are set forth in Part 8 of Remington's The Science and Practice of Pharmacy, 21st edition, 2005, Publisher: Lippincott Williams & Wilkins, which is incorporated herein by reference.

In some embodiments, compositions provided herein, e.g., compositions comprising a compound of Formula (I) or hydrate thereof, can further comprise epinephrine, levonordefrin, a salt of epinephrine, a salt of levonordefrin, or hydrate thereof. In some embodiments, the salt of epinephrine is epinephrine saccharinate, epinephrine acesulfamate, epinephrine glycyrrherinate, epinephrine mono-glycyrrhizinate, epinephrine tri-glycyrrhizinate, epinephrine vanillate, epinephrine ferrulate, epinephrine glycinate, epinephrine cinnamate, epinephrine enoxolone, epinephrine cyclamate, epinephrine steviol, epinephrine aspartamate, epinephrine di-glycyrrhinizinate, or epinephrine neotame. In some embodiments, the salt of levonordefrin is levonordefrin saccharinate, levonordefrin acesulfamate, levonordefrin glycyrrherinate, levonordefrin mono-glycyrrhizinate, levonordefrin tri-glycyrrhizinate, levonordefrin vanillate, levonordefrin ferrulate, levonordefrin glycinate, levonordefrin cinnamate, levonordefrin enoxolone, levonordefrin cyclamate, levonordefrin steviol, levonordefrin aspartamate, levonordefrin di-glycyrrhinizinate, or levonordefrin neotame. In some embodiments, epinephrine, levonordefrin, a salt of epinephrine, a salt of levonordefrin, or hydrate thereof comprises at least 0.000001% by weight of the total composition. In some embodiments, epinephrine, levonordefrin, a salt of epinephrine, a salt of levonordefrin, or hydrate thereof is administered in an effective amount ranging from about 0.000001% by weight to about 5% by weight, e.g., from about 0.000001% by weight to about 0.001% by weight of the total composition. In some embodiments, the salt of epinephrine, salt of levonordefrin, or hydrate thereof is generally administered in an effective amount of about 0.00001% by weight of the total composition.

In some embodiments, pharmaceutically acceptable excipients present in the compositions described herein, e.g., a composition comprising a compound of Formula (I) or hydrate thereof, can be, e.g., osmolality adjusting agents or pH adjusting agents. In some embodiments, the compositions described herein can comprise both osmolality adjusting agents and pH adjusting agents.

In some embodiments, the pH of a compound described herein can be at least 3.0, e.g., from about 3.5 to about 5.4. In other embodiments, the pH of a composition described herein, e.g., an aqueous compound formulated for injection, can be at least from about 3.0, e.g., from about 3.6 to about 4.4. In some embodiments, the pH of a compound described herein has a pH higher than a hydrochloride salt of the caine family, e.g., lidocaine hydrochloride.

### Methods of Use

### Local Anesthesia

Compounds described herein, e.g., a compound of Formula (I) or hydrate thereof, are formulated for use as local anesthetics. Thus, in an aspect, the present invention provides herein a method of suppressing pain experienced by a subject during a cosmetic, medical or dental procedure, comprising administering to the subject an effective amount of a composition comprising a compound of Formula (I) or hydrate thereof:

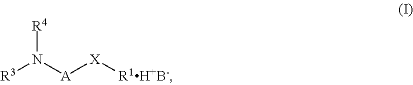

or hydrate thereof, wherein:

B is a sweetener (e.g., saccharinate, acesulfamate, glycyrrherinate, mono-glycyrrhizinate, tri-glycyrrhizinate, vanillate, ferrulate, glycinate, cinnamate, enoxolone, cyclamate, steviol, aspartamate, di-glycyrrhinizinate, neotame); X is —C(O)O—, —OC(O)—, —C(O)NR^{A}—, or —NR^{A}C(O)—, or —CH(OR^{A})—, wherein R^{A} is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};

R¹ is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};

each of R³ and R⁴ is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, and heteroaryl is independently substituted with 0-5 occurrences of R^{Z};

A is:

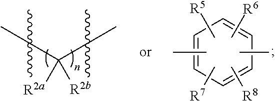

wherein: n is 1-5; each of R^{2a} and R^{2b} is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}, or if n is 1, R^{2a} or R^{2b} and R³ or R⁴, together with the atoms to which they are attached form a 3-8 membered ring independently substituted with 0-5 occurrences of R^{Z}; and each of R⁵, R⁶, R⁷, and R⁸ is independently hydrogen, alkyl (e.g., C₁-C₆ alkyl), C₁-C₆ alkoxy, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; and R^{Z} is halogen, C₁-C₆ alkyl, C₁-C₆ haloalkyl, C₁-C₆ heteroalkyl, —NHR^{Z1}, —NR^{Z1}R^{Z2}, —C(O)R^{Z1}, —C(O)R^{Z2}, —C(O)NR^{Z1}R^{Z2}, —NR^{Z1}C(O)R^{Z2}, —OR^{Z1}, —OR^{Z2}, cyano, or nitro, wherein R^{Z1} is hydrogen, C₁-C₆ alkyl, C₁-C₆ haloalkyl, C₁-C₆ heteroalkyl, hydroxyl, cyano, or nitro and R^{Z2} is carbocyclylalkyl, heterocycylalkyl, aralkyl or heteroaralkyl substituted with 0-5 occurrences of R^{Z1}.

In some embodiments, the composition is formulated for injection as described herein. In some embodiments, the composition is formulated as a lotion, ointment, or patch for transdermal administration as described herein. In some embodiments, the composition is administered intraorally, epidurally, intranasally, ocularly, transdermally, intramuscularly, subcutaneously, intramuscularly, or transmucosally. In some embodiments, administration of a composition described herein, e.g, a composition comprising a compound of Formula (I) by, e.g., intraoral administration, does not elicit an objectionable bitter taste experienced by a patient during, e.g., oral surgery. In some embodiments, the compounds described herein, e.g., a compound of Formula (I) or hydrate thereof, do not elicit pain or tissue damage associated with the lower pH of their corresponding hydrochloride salts and can therefore be more widely applied in medical procedures, e.g., epidural procedures, skin surgery, eye surgery, or biopsies, and cosmetic procedures, e.g., facial injections, hair transplants, or liposuction. In some embodiments, the pH of the compound of Formula (I) or hydrate thereof is at least 3.0, e.g. from about 3.5 to about 5.5. In some embodiments, the pH of a composition comprising a compound of Formula (I) or hydrate thereof is at least 3.0, e.g., from about 3.6 to about 5.5.

Compounds contemplated for use in local anesthesia as described herein can be salts comprising cations that function as the active local anesthetic, e.g., protonated caines, and anions capable of masking bitter tastes. In some embodiments, the compound for use as the local anesthetic can be, but is not limited to, lidocaine saccharinate, lidocaine acesulfamate, lidocaine glycyrrherinate, lidocaine mono-glycyrrhizinate, lidocaine tri-glycyrrhizinate, lidocaine vanillate, lidocaine ferrulate, lidocaine glycinate, lidocaine cinnamate, lidocaine enoxolone, lidocaine cyclamate, lidocaine steviol, lidocaine aspartamate, lidocaine di-glycyrrhinizinate, lidocaine neotame, tetracaine saccharinate, tetracaine acesulfamate, tetracaine glycyrrherinate, tetracaine mono-glycyrrhizinate, tetracaine tri-glycyrrhizinate, tetracaine vanillate, tetracaine ferrulate, tetracaine glycinate, tetracaine cinnamate, tetracaine enoxolone, tetracaine cyclamate, tetracaine steviol, tetracaine aspartamate, tetracaine di-glycyrrhinizinate, tetracaine neotame, bupivacaine saccharinate, bupivacaine acesulfamate, bupivacaine glycyrrherinate, bupivacaine mono-glycyrrhizinate, bupivacaine tri-glycyrrhizinate, bupivacaine vanillate, bupivacaine ferrulate, bupivacaine glycinate, bupivacaine cinnamate, bupivacaine enoxolone, bupivacaine cyclamate, bupivacaine steviol, bupivacaine aspartamate, bupivacaine di-glycyrrhinizinate, bupivacaine neotame, mepivacaine saccharinate, mepivacaine acesulfamate, mepivacaine glycyrrherinate, mepivacaine mono-glycyrrhizinate, mepivacaine tri-glycyrrhizinate, mepivacaine vanillate, mepivacaine ferrulate, mepivacaine glycinate, mepivacaine cinnamate, mepivacaine enoxolone, mepivacaine cyclamate, mepivacaine steviol, mepivacaine aspartamate, mepivacaine di-glycyrrhinizinate, mepivacaine neotame, articaine saccharinate, articaine acesulfamate, articaine glycyrrherinate, articaine mono-glycyrrhizinate, articaine tri-glycyrrhizinate, articaine vanillate, articaine ferrulate, articaine glycinate, articaine cinnamate, articaine enoxolone, articaine cyclamate, articaine steviol, articaine aspartamate, articaine di-glycyrrhinizinate, articaine neotame, prilocaine saccharinate, prilocaine acesulfamate, prilocaine glycyrrherinate, prilocaine mono-glycyrrhizinate, prilocaine tri-glycyrrhizinate, prilocaine vanillate, prilocaine ferrulate, prilocaine glycinate, prilocaine cinnamate, prilocaine enoxolone, prilocaine cyclamate, prilocaine steviol, prilocaine aspartamate, prilocaine di-glycyrrhinizinate, prilocaine neotame, procaine saccharinate, procaine acesulfamate, procaine glycyrrherinate, procaine mono-glycyrrhizinate, procaine tri-glycyrrhizinate, procaine vanillate, procaine ferrulate, procaine glycinate, procaine cinnamate, procaine enoxolone, procaine cyclamate, procaine steviol, procaine aspartamate, procaine di-glycyrrhinizinate, procaine neotame, oxybuprocaine saccharinate, oxybuprocaine acesulfamate, oxybuprocaine glycyrrherinate, oxybuprocaine mono-glycyrrhizinate, oxybuprocaine tri-glycyrrhizinate, oxybuprocaine vanillate, oxybuprocaine ferrulate, oxybuprocaine glycinate, oxybuprocaine cinnamate, oxybuprocaine enoxolone, oxybuprocaine cyclamate, oxybuprocaine steviol, oxybuprocaine aspartamate, oxybuprocaine di-glycyrrhinizinate, oxybuprocaine neotame, ropivacaine saccharinate, ropivacaine acesulfamate, ropivacaine glycyrrherinate, ropivacaine mono-glycyrrhizinate, ropivacaine tri-glycyrrhizinate, ropivacaine vanillate, ropivacaine ferrulate, ropivacaine glycinate, ropivacaine cinnamate, ropivacaine enoxolone, ropivacaine cyclamate, ropivacaine steviol, ropivacaine aspartamate, ropivacaine di-glycyrrhinizinate, ropivacaine neotame, cinchocaine saccharinate, cinchocaine acesulfamate, cinchocaine glycyrrherinate, cinchocaine mono-glycyrrhizinate, cinchocaine tri-glycyrrhizinate, cinchocaine vanillate, cinchocaine ferrulate, cinchocaine glycinate, cinchocaine cinnamate, cinchocaine enoxolone, cinchocaine cyclamate, cinchocaine steviol, cinchocaine aspartamate, cinchocaine di-glycyrrhinizinate, cinchocaine neotame, benzocaine saccharinate, benzocaine acesulfamate, benzocaine glycyrrherinate, benzocaine mono-glycyrrhizinate, benzocaine tri-glycyrrhizinate, benzocaine vanillate, benzocaine ferrulate, benzocaine glycinate, benzocaine cinnamate, benzocaine enoxolone, benzocaine cyclamate, benzocaine steviol, benzocaine aspartamate, benzocaine di-glycyrrhinizinate, benzocaine neotame, oxetacaine saccharinate, oxetacaine acesulfamate, oxetacaine glycyrrherinate, oxetacaine mono-glycyrrhizinate, oxetacaine tri-glycyrrhizinate, oxetacaine vanillate, oxetacaine ferrulate, oxetacaine glycinate, oxetacaine cinnamate, oxetacaine enoxolone, oxetacaine cyclamate, oxetacaine steviol, oxetacaine aspartamate, oxetacaine di-glycyrrhinizinate, or oxetacaine neotame. In some embodiments, the administered composition can comprise at least 0.0001% by weight of a compound of Formula (I) or hydrate thereof. In some embodiments, the administered composition, when formulated for injection, can comprise from about 1% by weight to about 3% by weight, e.g., 1% by weight, of a compound of Formula (I) or hydrate thereof. In some embodiments, the administered composition, when formulated for topical or transdermal administration, can comprise from about 1% by weight to about 10% by weight, e.g., about 5% by weight, of a compound of Formula (I) or hydrate thereof.

In some embodiments, the composition can further comprise epinephrine, levonordefrin, or a salt thereof and a pharmaceutically acceptable carrier. The epinephrine salt can be, but is not limited to, epinephrine saccharinate, epinephrine acesulfamate, epinephrine glycyrrherinate, epinephrine mono-glycyrrhizinate, epinephrine tri-glycyrrhizinate, epinephrine vanillate, epinephrine ferrulate, epinephrine glycinate, epinephrine cinnamate, epinephrine enoxolone, epinephrine cyclamate, epinephrine steviol, epinephrine aspartamate, epinephrine di-glycyrrhinizinate, epinephrine neotame. The levonordefrin salt can be, but is not limited to, levonordefrin saccharinate, levonordefrin acesulfamate, levonordefrin glycyrrherinate, levonordefrin mono-glycyrrhizinate, levonordefrin tri-glycyrrhizinate, levonordefrin vanillate, levonordefrin ferrulate, levonordefrin glycinate, levonordefrin cinnamate, levonordefrin enoxolone, levonordefrin cyclamate, levonordefrin steviol, levonordefrin aspartamate, levonordefrin di-glycyrrhinizinate, or levonordefrin neotame. In some embodiments, the administered composition comprises at least 0.000001% by weight of epinephrine, levonordefrin, a salt of epinephrine, a salt of levonodefrin, or hydrate thereof. In some embodiments, the administered composition comprises from about 0.000001% by weight to about 10% by weight of epinephrine, levonordefrin, a salt of epinephrine, a salt of levonodefrin, or hydrate thereof. In some embodiments, the administered composition comprises about 0.00001% by weight of epinephrine, levonordefrin, a salt of epinephrine, a salt of levonodefrin, or hydrate thereof.

### Methods of Making

In another aspect, also provided herein is a method for making the compound of Formula (I) or hydrate thereof, the method comprising dissolving the compound of Formula (II):

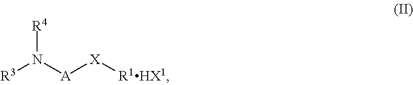

wherein: X¹ is —OH or a halide anion (e.g., chloride, bromide, or iodide); X is —C(O)O—, —OC(O)—, —C(O)NR^{A}—, or —NR^{A}C(O)—, or —CH(OR^{A})—, wherein R^{A} is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; R¹ is hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; each of R³ and R⁴ is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, and heteroaryl is independently substituted with 0-5 occurrences of R^{Z};

A is:

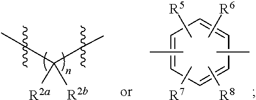

wherein: n is 1-5; each of R^{2a} and R^{2b} is independently hydrogen, alkyl (e.g., C₁-C₆ alkyl), carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}, or if n is 1, R^{2a} or R^{2b} and R³ or R⁴, together with the atoms to which they are attached form a 3-8 membered ring independently substituted with 0-5 occurrences of R^{Z}; and each of R⁵, R⁶, R⁷, and R⁸ is independently hydrogen, alkyl (e.g., C₁-C₆ alkyl), C₁-C₆ alkoxy, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; and R^{Z} is halogen, C₁-C₆ alkyl, C₁-C₆ haloalkyl, C₁-C₆ heteroalkyl, —NHR^{Z1}, —NR^{Z1}R^{Z2}, —C(O)R^{Z1}, —C(O)R^{Z2}, —C(O)NR^{Z1}R^{Z2}, —NR^{Z1}C(O)R^{Z2}, —OR^{Z1}, —OR^{Z2}, cyano, or nitro, wherein R^{Z1} is hydrogen, C₁-C₆ alkyl, C₁-C₆ haloalkyl, C₁-C₆ heteroalkyl, hydroxyl, cyano, or nitro and R^{Z2} is carbocyclylalkyl, heterocycylalkyl, aralkyl or heteroaralkyl substituted with 0-5 occurrences of R^{Z1};

provided that the following compounds or hydrates thereof are excluded:

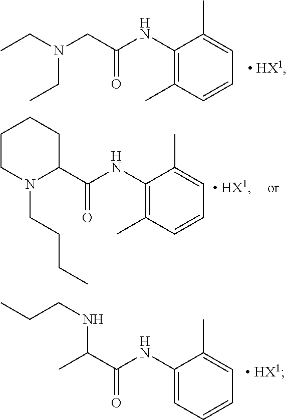

and an organic salt in a solvent, wherein a metal halide precipitates or is solubilized and the compound of Formula (I) or hydrate thereof remains solubilized.

In some embodiments, the organic salt can be, but is not limited to, sodium saccharinate, sodium acesulfamate, sodium glycyrrherinate, sodium mono-glycyrrhizinate, sodium tri-glycyrrhizinate, sodium vanillate, sodium ferrulate, sodium glycinate, sodium cinnamate, sodium enoxolone, sodium cyclamate, sodium steviol, sodium aspartamate, sodium di-glycyrrhinizinate, sodium neotame, potassium saccharinate, potassium acesulfamate, potassium glycyrrherinate, potassium mono-glycyrrhizinate, potassium tri-glycyrrhizinate, potassium vanillate, potassium ferrulate, potassium glycinate, potassium cinnamate, potassium enoxolone, potassium cyclamate, potassium steviol, potassium aspartamate, potassium di-glycyrrhinizinate, or potassium neotame. In some embodiments, X¹ is chloride. In some embodiments, X¹ is —OH. In some embodiments, the solvent is acetonitrile. Other examples of solvents include, but are not limited to, water, 1,4-dioxane, ethyl acetate, acetone, methanol, ethanol, isopropanol, butanol, acetone, 2-butanone, ethylene glycol, ethylene glycol monomethyl ether, 1,2-dimethoxyethane, and 2-methoxyethanol. In another embodiment, the method produces a compound of Formula (I) or hydrate thereof in high yield, e.g., from about 90% yield to about 100% yield.

### Abbreviations

MeCN: acetonitrile; MeOH: methanol; TLC: thin-layer chromatography; DMSO: dimethyl sulfoxide.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Materials and Methods

The compounds provided herein can be prepared from readily available starting materials using the following general methods and procedures. It will be appreciated that where typical or preferred process conditions (e.g., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, or high-performance liquid chromatography (HPLC). The following schemes are presented with details as to the preparation of representative compounds that have been listed herein. The compounds provided herein may be prepared from known or commercially available starting materials and reagents by one skilled in the art of organic synthesis.

### Animal Studies

Male Sprague Dawley® rats (200-300 g, Charles River) were housed in groups of two and were maintained in a standard 12-h light/dark cycle and testing was completed in the light portion of the cycle between 09:00-12:00. Animals were placed into the behavioural procedure room 30 min prior to testing to acclimate. When not in testing sessions, food and water were made available ad libitum. Animal testing procedures complied with the ethical guidelines and standards established by the University of Florida's Institutional Animal Care & Use Committee and with the Guide for Care and Use of Laboratory Animals (National Research Council Guide for the Care and Use of Laboratory Animals. Washington, D.C., National Academy Press; 1996.).

¹H-NMR reported herein (e.g., for the region between 6 (ppm) of about 0.5 ppm to about 8 ppm) will be understood to be an exemplary interpretation of the NMR spectrum (e.g., exemplary peak integratations) of a compound.

### Example 1. Syntheses of Caine Salts 3a-3h

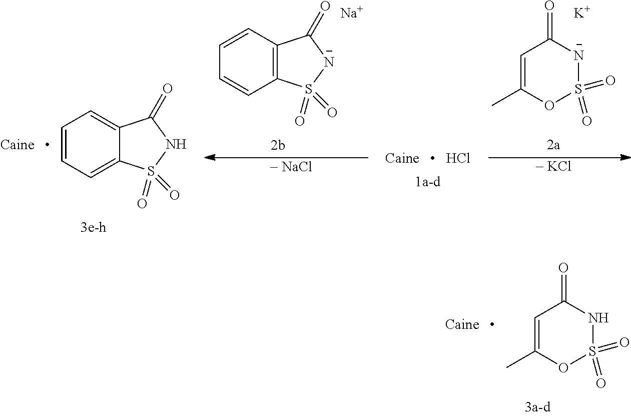

To the solution of a hydrochloride salt of a corresponding caines 1a-d (1.0 mmol) in MeCN (15 mL) equimolar quantity of the sweetener salt: (potassium 6-methyl-1,2,3-oxathiazin-4-olate 2,2-dioxide 0.185 g for 2a or sodium benzo[d]isothiazol-3-olate 1,1-dioxide 0.205 g for 2b) was added and the mixture was then stirred for 4h at the ultrasonic bath at 50° C. After the reaction was completed (followed by TLC), reaction mixture was filtered through the 22 micron membrane filter and the filtrate was taken to dryness. After that diethyl ether (3×25 mL) was added to product and it was evaporated to give products 3a-h in quantitative yields. Final products 3a-h are all water-soluble ionic conjugates that consist of the anion of a sweetener (acesulfamate or saccahine) and a representative of a caine family (mepivacaine, bupivacaine, prilocaine, articaine) as a cation.

Mepivacaine acesulfamate (3a)

White semisolid (97%, 0.396 g, 0.97 mmol). m.p. 84.0-86.0° C.; ¹H NMR (300 MHz, CD₃OD, δ) 7.21-7.09 (m, 3H), 5.53 (s, 1H), 4.17 (dd, J=11.9, 3.2 Hz, 1H), 3.54 (d, J=12.5 Hz, 1H), 3.18 (td, J=12.4, 3.1 Hz, 1H), 2.93 (d, J=1.1 Hz, 3H), 2.46-2.33 (m, 1H), 2.22 (d, J=0.9 Hz, 6H), 2.02 (t, J=1.0 Hz, 3H), 1.93 (s, 3H), 1.89-1.67 (m, 2H); ¹³C NMR (75 MHz, CD₃OD, δ) 173.2, 168.8, 164.4, 137.2, 134.7, 129.9, 129.5, 102.8, 68.8, 56.7, 43.6, 31.0, 24.6, 22.9, 20.4, 19.1. HRMS (ESI) calcd for C₁₅H₂₂N₂O [M+H]⁺ 246.3535, found 247.1800; HRMS (ESI) calcd for C₄H₄NO₄S [M−H]⁻ 161.9867, found 161.9873.

### Bupivacaine Acesulfamate (3b)

Colorless oil (96%, 0.432 g, 0.96 mmol)). ¹H NMR (300 MHz, CD₃OD, δ) 7.18-7.11 (m, 3H), 5.55 (s, 1H), 4.32 (d, J=11.6 Hz, 1H), 3.69 (d, J=12.2 Hz, 1H), 3.19 (dq, J=16.5, 11.0, 8.5 Hz, 3H), 2.42 (d, J=12.2 Hz, 1H), 2.24 (s, 6H), 2.04 (s, 3H), 1.99-1.70 (m, 7H), 1.41 (q, J=7.4 Hz, 2H), 0.99 (t, J=7.3 Hz, 3H); ¹³C NMR (75 MHz, CD₃OD, δ) 173.2, 169.0, 164.4, 137.2, 134.7, 129.9, 129.9, 129.5, 102.9, 57.8, 53.7, 31.0, 27.3, 24.4, 21.5, 21.5, 20.4, 19.1, 14.4. HRMS (ESI) calcd for C₁₈H₂₈N₂O [M+H]⁺ 288.2196, found 289.2262; HRMS (ESI) calcd for C₄H₄NO₄S [M−H]⁻ 161.9867, found 161.9872.

### Prilocaine Acesulfamate (3c)

White solid (98%, 0.375 g, 0.98 mmol)). m.p. 145.2-145.8° C.; ¹H NMR (300 MHz, CD₃OD, δ) 7.38-7.34 (m, 1H), 7.25-7.15 (m, 3H), 5.52 (s, 1H), 4.20 (q, J=0.6 Hz, 1H), 3.12-2.92 (m, 2H), 2.26 (s, 3H), 2.02 (s, 3H), 1.71 (dd, J=23.5, 7.3 Hz, 2H), 1.66 (d, J=0.6 Hz, 3H), 1.02 (t, J=7.4 Hz, 3H); ¹³C NMR (75 MHz, CD₃OD, δ) 173.0, 169.6, 164.2, 136.1, 134.6, 132.0, 132.0, 128.3, 127.7, 127.1, 102.5, 57.9, 49.39, 21.1, 18.4, 17.2, 11.5. HRMS (ESI) calcd for C₁₃H₂₀N₂O [M+H]⁺ 220.1576, found 221.1645; HRMS (ESI) calcd for C₄H₄NO₄S [M−H]⁻ 161.9867, found 161.9875.

### Articaine Acesulfamate (3d)

White solid (98%, 0.438 g, 0.98 mmol). m.p. 166.2-169.8° C.; ¹H NMR (300 MHz, CD₃OD, δ) 7.41-7.40 (m, 1H), 3.83-3.82 (m 4H), 3.08-3.04 (m, 2H), 2.13 (t, J=1.4 Hz, 3H), 2.04-2.03 (m, 4H), 1.78-1.70 (m, 5H), 1.06-1.01 (m, 3H); ¹³C NMR (75 MHz, CD₃OD, δ) 173.3, 169.8, 164.5, 163.6, 140.2, 138.5, 128.75, 125.4, 102.8, 58.1, 53.0, 49.78, 21.3, 20.4, 17.4, 14.6, 11.8. HRMS (ESI) calcd for C₁₃H₂₀N₂O₃S [M+H]⁺ 284.1195, found 285.1261; HRMS (ESI) calcd for C₄H₄NO₄S [M−H]⁻ 161.9867, found 161.9875.

### Mepivacaine Saccharinate (3e)

Colorless oil (95%, 0.407 g, 0.95 mmol); ¹H NMR (300 MHz, CD₃OD, δ) 7.82-7.76 (m, 2H), 7.75-7.65 (m, 2H), 7.18-7.09 (m, 3H), 4.20 (dd, J=11.8, 3.3 Hz, 1H), 3.53 (d, J=12.4 Hz, 1H), 3.18 (td, J=12.5, 3.1 Hz, 1H), 2.94 (s, 3H), 2.39 (dd, J=14.0, 3.5 Hz, 1H), 2.39 (d, J=3.4 Hz, 1H), 2.27 (s, 6H), 1.95 (d, J=12.6 Hz, 2H), 1.90-1.60 (m, 2H); ¹³C NMR (75 MHz, CD₃OD, δ) 172.2, 168.5, 145.7, 136.9, 135.1, 134.4, 134.1, 133.7, 129.5, 129.2, 124.66, 121.2, 68.4, 56.4, 43.3, 30.6, 24.2, 22.5, 18.8. HRMS (ESI) calcd for C₁₅H₂₂N₂O [M+H]⁺ 246.1727, found 247.1799; HRMS (ESI) calcd for C₇H₄NO₃S [M−H]⁻ 181.9917, found 181.9924.

### Bupivacaine Saccharinate (3f)

White solid (97%, 0.456 g, 0.97 mmol). m.p. 162.4-164.8° C. ¹H NMR (300 MHz, DMSO-d₆, δ) 10.17 (s, 1H), 9.70 (s, 1H), 7.64-7.57 (m, 1H), 7.58 (s, 1H), 7.13 (m, 3H), 4.07 (s, 1H), 3.52 (d, J=12.4 Hz, 1H), 3.11-3.01 (m, 4H), 2.15 (s, 6H), 1.92-1.52 (m, 7H), 1.31 (q, J=7.4 Hz, 2H), 0.90 (t, J=7.3 Hz, 3H); ¹³C NMR (75 MHz, CD₃OD, δ) 172.4, 168.7, 145.8, 136.8, 136.8, 135.2, 134.4, 134.07, 133.7, 129.6, 129.2, 124.7, 121.2, 57.4, 53.3, 30.7, 27.0, 24.0, 21.2, 18.7, 14.1. HRMS (ESI) calcd for C₁₈H₂₈N₂O [M+H]⁺ 288.2196, found 289.2266; HRMS (ESI) calcd for C₇H₄NO₃S [M−H]⁻ 181.9917, found 181.9924.

### Prilocaine Saccharinate (3g)

White solid (96%, 0.386 g, 0.96 mmol). m.p. 122.0-123.4° C.; ¹H NMR (300 MHz, CD₃OD, δ) 7.81-7.69 (m, 3H), 7.41-7.38 (dd, J=7.3, 2.0 Hz, 1H), 7.30-7.20 (m, 4H), 4.22 (q, J=7.0 Hz, 1H), 3.11-3.05 (m, 2H), 2.29 (s, 3H), 1.78 (m, 5H), 1.06 (t, J=7.4 Hz, 3H); ¹³C NMR (75 MHz, CD₃OD, δ) 172.7, 169.9, 146.1, 136.4, 135.5, 134.9, 134.4, 134.0, 132.4, 128.6, 128.1, 127.4, 125.0, 121.6, 58.3, 49.7, 21.5, 18.7, 17.6, 11.9. HRMS (ESI) calcd for C₁₃H₂₀N₂O [M+H]⁺220.1576, found 221.1646; HRMS (ESI) calcd for C₇H₄NO₃S [M−H]⁻ 181.9917, found 181.9923.

### Articaine Saccharinate (3h)

Yellow oil (99%, 0.462 g, 0.99 mmol). ¹H NMR (300 MHz, CD₃OD, δ) 8.79-8.76 (m, 2H), 8.69-8.66 (m, 2H), 8.39 (t, J=1.1 Hz, 1H), 5.31 (d, J=6.0 Hz, 1H), 4.81-4.80 (m, 3H), 4.10-4.05 (m, 2H), 3.11 (dt, J=2.1, 1.0 Hz, 3H), 2.73 (d, J=7.0 Hz, 5H), 2.01 (t, J=7.4 Hz, 3H); ¹³C NMR (75 MHz, CD₃OD, δ) 172.7, 169.8, 163.5, 145.9, 140.1, 138.4, 138.4, 134.3, 133.9, 128.8, 124.9, 124.9, 121.5, 58.1, 53.0, 49.8, 21.3, 17.5, 14.6, 11.8. HRMS (ESI) calcd for C₁₃H₂₀N₂O₃S [M+H]⁺ 284.1195, found 285.1261; HRMS (ESI) calcd for C₇H₄NO₃S [M−H]⁻ 181.9917, found 181.9925.

### Example 2. Syntheses of Salts of Epinephrine Acesulfamate (4a) and Epinephrine Saccharinate (4b)

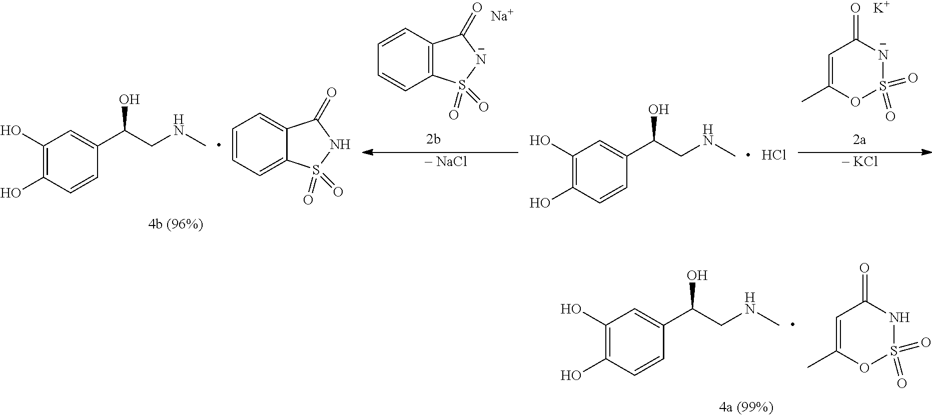

Syntheses of 5a and 5b: To the solution of epinephrine hydrochloride (1.0 mmol, 220 mg) in MeCN (15 mL) equimolar quantity of sweetener potassium salt (sodium 6-methyl-1,2,3-oxathiazin-4-olate 2,2-dioxide 0.185 g for 2a and sodium benzo[d]isothiazol-3-olate 1,1-dioxide 0.205 g for 2b) was added and the mixture was then stirred for 4h at the ultrasonic bath at 50° C. After the reaction was completed (followed by TLC), reaction mixture was filtered through the 22 micron membrane filter and the filtrate was taken to dryness. After that diethyl ether (3×25 mL) was added to product and it was evaporated to give products 5a and 5b in quantitative yields.

### Epinephrine Acesulfamate (4a)

Colorless oil (99%, 0.343 g, 0.99 mmol). ¹H NMR (300 MHz, CD₃OD, δ) 6.88 (s, 1H), 6.79-6.72 (m, 2H), 5.53 (s, 1H), 4.83-4.79 (m, 1H), 3.18-3.04 (m, 2H), 2.74 (s, 3H), 2.05-2.04 (m 3H); ¹³C NMR (75 MHz, CD₃OD, δ) 172.0, 163.2, 145.7, 145.6, 132.8, 117.7, 115.6, 113.4, 100.4, 69.0, 55.9, 33.0, 19.0. HRMS (ESI) calcd for C₉H₁₄NO₃ [M+H]⁺ 184.0968, found 184.0964; HRMS (ESI) calcd for C₄H₄NO₄S [M−H]⁻ 161.9867, found 161.9866.

### Epinephrine Saccharinate (4b)

Yellow solid (96%, 0.352 g, 0.96 mmol). m.p. 82.0-83.2° C. ¹H NMR (300 MHz, CD₃OD, δ) 7.72-7.63 (m, 2H), 6.93 (d, J=2.0 Hz, 1H), 6.82-6.72 (m, 2H), 4.88 (dd, J=9.4, 3.9 Hz, 3H), 2.77 (s, 3H), 2.02 (d, J=0.6 Hz, 3H); ¹³C NMR (75 MHz, CD₃OD, δ) 171.3, 145.4, 145.3, 144.2, 133.7, 132.9, 132.6, 132.5, 123.5, 120.0, 117.7, 115.5, 113.3, 68.8, 55.7, 32.9. HRMS (ESI) calcd for C₉H₁₄NO₃ [M+H]⁺ 184.0968, found 184.0966; HRMS (ESI) calcd for C₇H₄NO₃S [M−H]⁻ 181.9917, found 181.9918.

### Example 3. Syntheses of Lidocaine Vanillate (5a) and Lidocaine Mono-Glycyrrhizinate (5b)

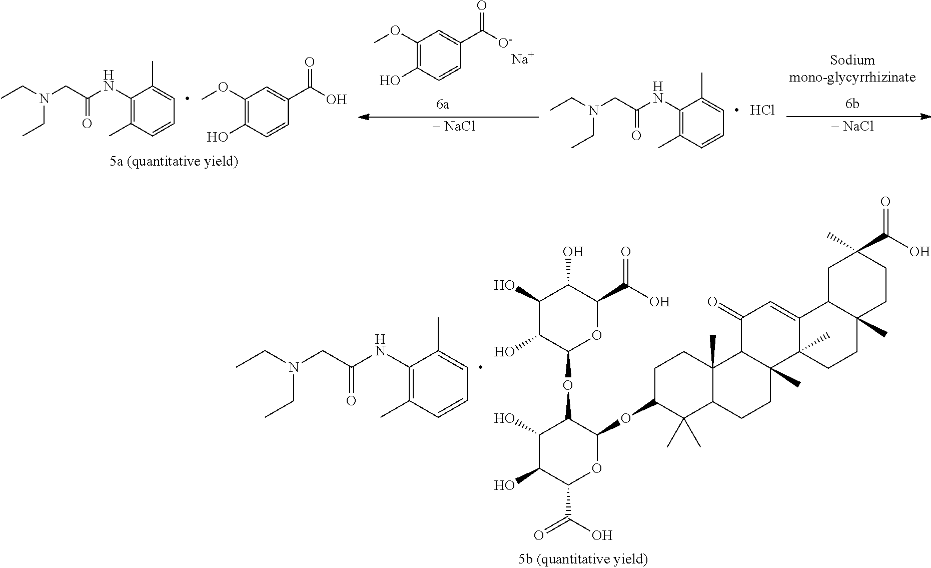

Sodium vanillate (6a) or sodium mono-glycyrrhizinate (6b) was mixed with equimolar amounts of lidocaine hydrochloride for four hours 50° C. in 1:1 MeOH/H₂O. After the solvent mixture was evaporated, the crude reaction mixture was dissolved in MeCN and sodium chloride was filtered off. Evaporation of MeCN yielded lidocaine vannilate (5a) as a light yellow solid (97% yield) or lidocaine mono-glycyrrhizinate (5b) as an off-white semisolid. (96% yield).

### Lidocaine Vanillate (5a)

¹H NMR (400 MHz, CD₃OD, δ) 7.42-7.37 (m, 3H), 6.95 (d, J=1.2 Hz, 2H), 6.65 (d, J=10.8 Hz, 2H), 3.72 (d, J=2.0 Hz, 2H), 2.75 (q, J=9.6 Hz, 4H), 2.07 (s, 6H), 1.10-1.04 (m, 6H). ¹³C NMR (75 MHz, CD₃OD, δ) 169.9, 150.4, 147.1, 135.4, 133.7, 127.8, 127.2, 123.6, 114.3, 114.2, 112.5, 55.6, 55.0, 46.9, 17.3, 10.5.

### Lidocaine Mono-Glycyrrhizinate (5b)

¹H NMR (400 MHz, CD₃OD, δ) 7.10 (d, J=3.6 Hz, 2H), 5.98 (br s, 1H), 3.81-3.73 (m, 2H), 3.21 (s, 1H), 3.11-3.06 (m, 2H), 2.84-2.76 (m, 4H), 2.45 (s, 1H), 2.21 (s, 6H), 2.04-1.83 (m, 3H), 1.74-1.69 (m, 4H), 1.41 (d, J=7.6 Hz, 4H), 1.28-1.17 (m, 16H), 0.99 (s, 3H), 0.83 (s, 3H), 0.8 (s, 3H). ¹³C NMR (75 MHz, CD₃OD, δ) 202.7, 181.1, 173.1, 136.8, 129.3, 129.1, 128.6, 79.5, 71.1, 63.3, 57.7, 56.3, 50.1, 45.3, 44.8, 42.8, 40.5, 39.3, 38.5, 36.5, 34.0, 33.1, 32.3, 29.4, 29.0, 28.8, 27.9, 27.8, 27.6, 26.9, 25.5, 24.0, 19.4, 18.7, 17.1, 16.5, 12.5.

### Example 4. Syntheses of Oxybuprocaine Saccharinate (7a) and Oxybuprocaine Acesulfamate (7b)

### Oxybuprocaine Saccharinate

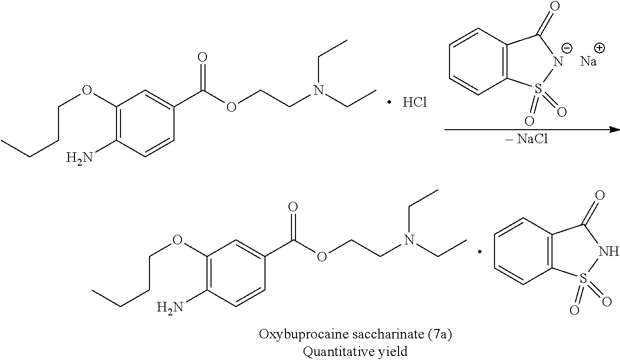

To a solution of oxybuprocaine hydrochloride (73 mg, 0.21 mmol) in 2 mL of ethanol was added the suspension of sodium saccharinate (43 mg, 0.21 mmol, 1 eq) in 2 mL of ethanol and reaction mixture was stirred at room temperature overnight.

The reaction mixture was then filtered and evaporated, the residue was dissolved in 3 mL of acetonitrile and after filtration and evaporation the product 7a was obtained as an off-white semisolid (103 mg, quantitative yield).

¹H NMR (500 MHz, CD₃CN, δ) 7.75 (m, 1H); 7.65 (m, 3H); 7.5 (d, 1H); 7.4 (s, 1H); 6.65 (d, 1H); 4.8 (br. s, 2H); 4.65 (t, 2H); 4.05 (t, 2H); 3.55 (t, 2H); 3.35 (q, 4H); 2.0 (m, 4H); 1.8 (m, 2H); 1.55 (m, 2H); 1.4 (t, 6H); 1.0 (t, 3H).

### Oxybuprocaine Acesulfamate

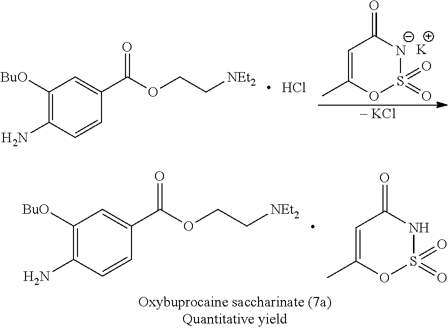

To a solution of oxybuprocaine hydrochloride (48 mg, 0.139 mmol) in 2 mL of ethanol was added a suspension of potassium acesulfame (28 mg, 0.139 mmol, 1 equivalent) in 2 mL of ethanol, and the reaction mixture was stirred at room temperature overnight.

The reaction mixture was then filtered and the solvent evaporated; the residue was dissolved in 3 mL of acetonitrile and after filtration and evaporation the product 7b was obtained as a yellow oil (67 mg, quantitative yield).

¹H NMR (500 MHz, CD₃CN, δ) 7.55 (d, 1H); 7.45 (s, 1H); 6.75 (d, 1H); 5.4 (s, 1H); 4.8 (broad s, 2H); 4.6 (t, 2H); 4.1 (t, 2H); 3.45 (t, 2H); 3.25 (q, 4H); 2.0 (m, 10H); 1.8 (m, 2H); 1.6 (m, 2H); 1.4 (t, 6H); 1.0 (t, 3H)

### Example 5. pH of Exemplary Salts

The pH values of saccharinate and acesulfamate caine salts 3a-3h with respect to concentration (conc.) as a weight-by volume percentage (% w/v) are shown in Table 1 below.

TABLE 1

| | Conc. (% w/v) | pH |
|---|---|---|
| Sodium saccharinate | 10% | 5.63 |
| | 5% | 6.09 |
| | 2% | 6.19 |
| Potassium acesulfamate | 10% | 5.78 |
| | 5% | 6.33 |
| | 2% | 5.98 |
| Mepivacaine hydrochloride | 10% | 3.73 |
| | 5% | 3.77 |
| | 2% | 4.53 |
| Mepivacaine saccharinate | 10% | 3.95 |
| | 5% | 4.06 |
| | 2% | 4.23 |
| Mepivacaine acesulfamate | 10% | 4.00 |
| | 5% | 4.87 |
| | 2% | 4.91 |
| Bupivacaine hydrochloride | 10% | 4.23 |
| | 5% | 4.81 |
| | 2% | 5.22 |
| Bupivacaine saccharinate | 10% | 3.84 |
| | 5% | 3.66 |
| | 2% | 5.19 |
| Bupivacaine acesulfamate | 10% | 4.25 |
| | 5% | 5.13 |
| | 2% | 5.38 |
| Lidocaine hydrochloride | 10% | 3.94 |
| | 5% | 4.53 |
| | 2% | 4.65 |
| Lidocaine acesulfamate | 10% | 4.84 |
| | 5% | 5.08 |
| | 2% | 5.26 |
| Lidocaine saccharinate | 10% | 4.31 |
| | 5% | 4.41 |
| | 2% | 4.51 |
| Prilocaine hydrochloride | 10% | 2.10 |
| | 5% | 3.42 |
| | 2% | 4.60 |
| Prilocaine saccharinate | 10% | 4.47 |
| | 5% | 4.96 |
| | 2% | 5.04 |
| Prilocaine acesulfamate | 10% | 4.71 |
| | 5% | 5.00 |
| | 2% | 5.16 |
| Epinephrine hydrochloride | 10% | 2.72 |
| | 5% | 3.91 |
| | 2% | 4.84 |
| Epinephrine saccharinate | 10% | 3.57 |
| | 5% | 4.03 |
| | 2% | 4.41 |
| Epinephrine acesulfamate | 10% | 4.01 |
| | 5% | 4.14 |
| | 2% | 4.34 |
| Articaine hydrochloride | 10% | 4.22 |
| | 5% | 4.62 |
| | 2% | 4.78 |
| Articaine saccharinate | 10% | 3.83 |
| | 5% | 3.95 |
| | 2% | 4.15 |
| Articaine acesulfamate | 10% | 3.50 |
| | 5% | 3.79 |
| | 2% | 3.95 |

### Example 6. Responses of Rats to Pain after Administration of a Caine Salt

1% by weight solutions of 3a-3h were injected into the hindpaw of rats and withdrawal latency to a thermal stimulus was recorded 10 minutes post-injection. Response to hindpaw heat pain was determined by placing unrestrained rats on a clear glass platform under a small plastic cage and rats habituated for 5 min. A radiant heat source was aimed directly under the ventral hindpaw surface and the time to paw withdrawal was recorded. Baseline responses were obtained under naïve conditions (e.g., no injection), while post-treatment effects of caine salts 3a-3h in the respective vehicle (water) were assessed for 10 minutes following injection (100 μl). A cutoff of 32 seconds was used to prevent tissue damage. FIG. 2 shows that caine salts 3a, 3b, 3c, 3d, 3e, 3f, and 3h (N=10 animals/group) produced a significant increase in latency time as compared to the naïve animals, indicating that the indicated caine salts were effective in inhibiting pain.

### Example 7. Palatability Assessments of Caine Salts 3a-3h

Rats were food-fasted (12-15 hours) prior to testing. Rats were then placed in an Orofacial Pain Assessment Device (OPAD, Stoelting Company, Wood Dale, Ill.) which consisted of a holding chamber and a bottle that contained the various test solutions. Animals (N=5/solution) were placed in the holding chamber and allowed access to the solution for 7 minutes and the number of solution licking events were automatically recorded. FIG. 2 shows that rats successfully consumed the different caine salt solutions.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 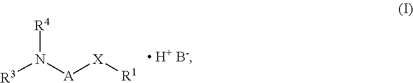
or hydrate thereof, wherein:
B is a sweetener;
X is —C(O)O—, —OC(O)—, —C(O)NR^{A}—, or —NR^{A}C(O)—, or —CH(OR^{A})—, wherein R^{A} is hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};
R¹ is hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};
each of R³ and R⁴ is independently hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, and heteroaryl is independently substituted with 0-5 occurrences of R^{Z};
A is: 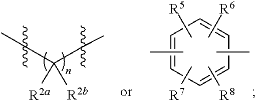
wherein:
n is 1-5;
each of R^{2a} and R^{2b} is independently hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}, or
if n is 1, R^{2a} or R^{2b} and R³ or R⁴, together with the atoms to which they are attached form a 3-8 membered ring independently substituted with 0-5 occurrences of R^{Z}; and
each of R⁵, R⁶, R⁷, and R⁸ is independently hydrogen, alkyl, C₁-C₆ alkoxy, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; and
R^{Z} is halogen, C₁-C₆ alkyl, C₁-C₆ haloalkyl, C₁-C₆ heteroalkyl, —NHR^{Z1}, —NR^{Z1}R^{Z2}, —C(O)R^{Z1}, —C(O)R^{Z2}, —C(O)NR^{Z1}R^{Z2}, —NR^{Z1}C(O)R^{Z2}, —OR^{Z1}, —OR^{Z2}, cyano, or nitro, wherein R^{Z1} is hydrogen, C₁-C₆ alkyl, C₁-C₆ haloalkyl, C₁-C₆ heteroalkyl, hydroxyl, cyano, or nitro and R^{Z2} is carbocyclylalkyl, heterocycylalkyl, aralkyl or heteroaralkyl substituted with 0-5 occurrences of R^{Z1};
provided that the compound or hydrate thereof is not lidocaine saccharinate, lidocaine acesulfamate, bupivacaine saccharinate, bupivacaine acesulfamate, prilocaine saccharinate, prilocaine acesulfamate, procaine saccharinate, cinchocaine saccharinate, or benzocaine saccharinate.

2. The compound of claim 1 or hydrate thereof, wherein the compound is a compound of Formula (I-A): 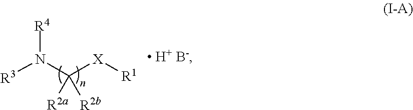
provided that the compound is not lidocaine saccharinate, lidocaine acesulfamate, bupivacaine saccharinate, bupivacaine acesulfamate, prilocaine saccharinate, prilocaine acesulfamate, procaine saccharinate, or cinchocaine saccharinate.

3. The compound of claim 1 or hydrate thereof, wherein the compound is a compound of Formula (I-B): 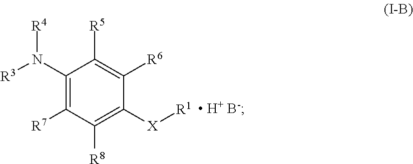
provided that the compound is not benzocaine saccharinate.

4. The compound of claim 1 or hydrate thereof, wherein B is saccharinate, acesulfamate, glycyrrherinate, mono-glycyrrhizinate, tri-glycyrrhizinate, vanillate, ferrulate, glycinate, cinnamate, enoxolone, cyclamate, steviol, aspartamate, di-glycyrrhinizinate, neotame

5. The hydrate of claim 1, wherein the hydrate is a monohydrate.

6. The compound of claim 1, wherein X is —C(O)NR^{A}— or —NR^{A}C(O)—.

7. The compound of claim 1 or hydrate thereof, wherein B is saccharinate or acesulfamate and R² and R³ are not —CH₂CH₃.

8. The compound of claim 1 or hydrate thereof, wherein each of R^{2a} and R^{2b} is independently hydrogen.

9. The compound of claim 1 or hydrate thereof, wherein R^{2a} and R^{2b} are hydrogen.

10. The compound of claim 1 or hydrate thereof, wherein each of R^{2a} and R^{2b} is independently hydrogen or alkyl independently substituted with 0-5 occurrences of R^{Z} and n is 1.

11. The compound of claim 1 or hydrate thereof, wherein each of R³ and R⁴ is independently hydrogen or alkyl independently substituted with 0-5 occurrences of R^{Z}.

12. The compound of claim 1 or hydrate thereof, wherein each of R³ and R⁴ is independently hydrogen or —CH₃.

13. The compound of claim 1 or hydrate thereof, wherein if n is 1, R³ or R⁴ and R^{2a} or R^{2b} together with the atoms to which they are attached form a 6-membered ring substituted with 0-5 occurrences of R^{Z}.

14. The compound of claim 3 or hydrate thereof, wherein at least one of R⁵, R⁶, R⁷, and R⁸ is not hydrogen.

15. The compound of claim 3 or hydrate thereof, wherein R³, R⁴, R⁵, R⁶, R⁷, and R⁸ are hydrogen.

16. The compound of claim 3 or hydrate thereof, wherein R³ and R⁴ are hydrogen and X is —C(O)O—.

17. The compound of claim 1 or hydrate thereof, wherein the compound is a compound of Formula (I-C), 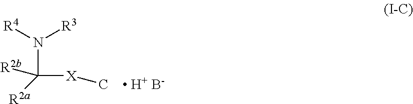
wherein C is a 5-10 membered ring substituted with 0-5 occurrences of R^{Z};
provided that the compound or hydrates thereof is not: lidocaine saccharinate, lidocaine acesulfamate, bupivacaine saccharinate, bupivacaine acesulfamate, cinchocaine saccharinate, prilocaine saccharinate, or prilocaine acesulfamate.

18. The compound of claim 17 or hydrate thereof, wherein C is, 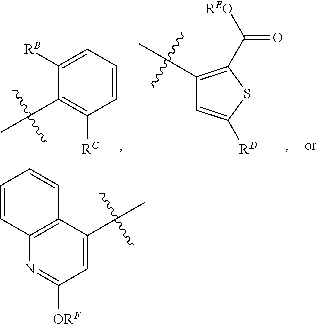
wherein each of R^{B}, R^{C}, R^{D}, R^{E}, or R^{F} is independently hydrogen or C₁-C₆ alkyl.

19. The compound of claim 17 or hydrate thereof, wherein R³ is n-butyl and R^{2a} and R⁴, together with the atoms to which they are attached form a 6-membered ring substituted with 0-5 occurrences of R^{Z}.

20. The compound of claim 17 or hydrate thereof, wherein R³ is methyl and R^{2a} and R⁴, together with the atoms to which they are attached form a 6-membered ring substituted with 0-5 occurrences of R^{Z}.

21. The compound of claim 17 or hydrate thereof, wherein R^{2a} is methyl and R⁴ is n-propyl.

22. The compound of claim 1 or hydrate thereof, wherein the salt is represented by Formula (I-D): 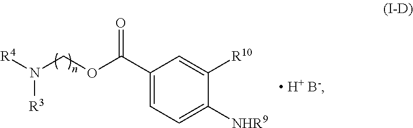
wherein R⁹ is hydrogen or C₁-C₆ alkyl and R¹⁰ is hydrogen or C₁-C₆ alkoxy;
provided that the compound is not procaine saccharinate.

23. The compound of claim 22 or hydrate thereof, wherein R³ and R⁴ are —CH₂CH₃.

24. The compound of claim 22 or hydrate thereof, wherein n is 2.

25. The compound of claim 22, wherein R³ and R⁴ are —CH₂CH₃, R⁹ is hydrogen or C₁-C₆ alkyl, and R¹⁰ is hydrogen or C₁-C₆ alkoxy.

26. The compound of claim 1 or hydrate thereof, wherein the salt is represented by Formula (I-E): 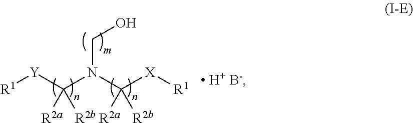
wherein m is 1, 2, 3, or 4 and Y is —NR^{A}C(O)— or —C(O)NR^{A}—.

27. The compound of claim 26 or hydrate thereof, wherein X is —C(O)NR^{A1}— and Y is —NR^{A1}C(O)—.

28. The compound of claim 26 or hydrate thereof, wherein R¹ is aralkyl.

29. The compound of claim 26 or hydrate thereof, wherein R^{2a} and R^{2b} are hydrogen.

30. The compound of claim 26 or hydrate thereof, wherein R^{2a} and R^{2b} are hydrogen and R is aralkyl.

31. The compound of claim 26 or hydrate thereof, wherein n is 1 and m is 1.

32. The compound of claim 1 or hydrate thereof, wherein the compound is lidocaine glycyrrherinate, lidocaine mono-glycyrrhizinate, lidocaine tri-glycyrrhizinate, lidocaine vanillate, lidocaine ferrulate, lidocaine glycinate, lidocaine cinnamate, lidocaine enoxolone, lidocaine cyclamate, lidocaine steviol, lidocaine aspartamate, lidocaine di-glycyrrhinizinate, lidocaine neotame, tetracaine saccharinate, tetracaine acesulfamate, tetracaine glycyrrherinate, tetracaine mono-glycyrrhizinate, tetracaine tri-glycyrrhizinate, tetracaine vanillate, tetracaine ferrulate, tetracaine glycinate, tetracaine cinnamate, tetracaine enoxolone, tetracaine cyclamate, tetracaine steviol, tetracaine aspartamate, tetracaine di-glycyrrhinizinate, tetracaine neotame, bupivacaine glycyrrherinate, bupivacaine mono-glycyrrhizinate, bupivacaine tri-glycyrrhizinate, bupivacaine vanillate, bupivacaine ferrulate, bupivacaine glycinate, bupivacaine cinnamate, bupivacaine enoxolone, bupivacaine cyclamate, bupivacaine steviol, bupivacaine aspartamate, bupivacaine di-glycyrrhinizinate, bupivacaine neotame, mepivacaine saccharinate, mepivacaine acesulfamate, mepivacaine glycyrrherinate, mepivacaine mono-glycyrrhizinate, mepivacaine tri-glycyrrhizinate, mepivacaine vanillate, mepivacaine ferrulate, mepivacaine glycinate, mepivacaine cinnamate, mepivacaine enoxolone, mepivacaine cyclamate, mepivacaine steviol, mepivacaine aspartamate, mepivacaine di-glycyrrhinizinate, mepivacaine neotame, articaine saccharinate, articaine acesulfamate, articaine glycyrrherinate, articaine mono-glycyrrhizinate, articaine tri-glycyrrhizinate, articaine vanillate, articaine ferrulate, articaine glycinate, articaine cinnamate, articaine enoxolone, articaine cyclamate, articaine steviol, articaine aspartamate, articaine di-glycyrrhinizinate, articaine neotame, prilocaine glycyrrherinate, prilocaine mono-glycyrrhizinate, prilocaine tri-glycyrrhizinate, prilocaine vanillate, prilocaine ferrulate, prilocaine glycinate, prilocaine cinnamate, prilocaine enoxolone, prilocaine cyclamate, prilocaine steviol, prilocaine aspartamate, prilocaine di-glycyrrhinizinate, prilocaine neotame, procaine acesulfamate, procaine glycyrrherinate, procaine mono-glycyrrhizinate, procaine tri-glycyrrhizinate, procaine vanillate, procaine ferrulate, procaine glycinate, procaine cinnamate, procaine enoxolone, procaine cyclamate, procaine steviol, procaine aspartamate, procaine di-glycyrrhinizinate, procaine neotame, oxybuprocaine saccharinate, oxybuprocaine acesulfamate, oxybuprocaine glycyrrherinate, oxybuprocaine mono-glycyrrhizinate, oxybuprocaine tri-glycyrrhizinate, oxybuprocaine vanillate, oxybuprocaine ferrulate, oxybuprocaine glycinate, oxybuprocaine cinnamate, oxybuprocaine enoxolone, oxybuprocaine cyclamate, oxybuprocaine steviol, oxybuprocaine aspartamate, oxybuprocaine di-glycyrrhinizinate, oxybuprocaine neotame, ropivacaine saccharinate, ropivacaine acesulfamate, ropivacaine glycyrrherinate, ropivacaine mono-glycyrrhizinate, ropivacaine tri-glycyrrhizinate, ropivacaine vanillate, ropivacaine ferrulate, ropivacaine glycinate, ropivacaine cinnamate, ropivacaine enoxolone, ropivacaine cyclamate, ropivacaine steviol, ropivacaine aspartamate, ropivacaine di-glycyrrhinizinate, ropivacaine neotame, cinchocaine acesulfamate, cinchocaine glycyrrherinate, cinchocaine mono-glycyrrhizinate, cinchocaine tri-glycyrrhizinate, cinchocaine vanillate, cinchocaine ferrulate, cinchocaine glycinate, cinchocaine cinnamate, cinchocaine enoxolone, cinchocaine cyclamate, cinchocaine steviol, cinchocaine aspartamate, cinchocaine di-glycyrrhinizinate, cinchocaine neotame, benzocaine acesulfamate, benzocaine glycyrrherinate, benzocaine mono-glycyrrhizinate, benzocaine tri-glycyrrhizinate, benzocaine vanillate, benzocaine ferrulate, benzocaine glycinate, benzocaine cinnamate, benzocaine enoxolone, benzocaine cyclamate, benzocaine steviol, benzocaine aspartamate, benzocaine di-glycyrrhinizinate, benzocaine neotame, oxetacaine saccharinate, oxetacaine acesulfamate, oxetacaine glycyrrherinate, oxetacaine mono-glycyrrhizinate, oxetacaine tri-glycyrrhizinate, oxetacaine vanillate, oxetacaine ferrulate, oxetacaine glycinate, oxetacaine cinnamate, oxetacaine enoxolone, oxetacaine cyclamate, oxetacaine steviol, oxetacaine aspartamate, oxetacaine di-glycyrrhinizinate, or oxetacaine neotame.

33. The compound of claim 1 or hydrate thereof, wherein the compound is epinephrine saccharinate, epinephrine acesulfamate, epinephrine glycyrrherinate, epinephrine mono-glycyrrhizinate, epinephrine tri-glycyrrhizinate, epinephrine vanillate, epinephrine ferrulate, epinephrine glycinate, epinephrine cinnamate, epinephrine enoxolone, epinephrine cyclamate, epinephrine steviol, epinephrine aspartamate, epinephrine di-glycyrrhinizinate, epinephrine neotame, levonordefrin saccharinate, levonordefrin acesulfamate, levonordefrin glycyrrherinate, levonordefrin mono-glycyrrhizinate, levonordefrin tri-glycyrrhizinate, levonordefrin vanillate, levonordefrin ferrulate, levonordefrin glycinate, levonordefrin cinnamate, levonordefrin enoxolone, levonordefrin cyclamate, levonordefrin steviol, levonordefrin aspartamate, levonordefrin di-glycyrrhinizinate, or levonordefrin neotame.

34. A composition comprising the compound of claim 1 or hydrate thereof and a pharmaceutically acceptable carrier.

35. The composition of claim 34, further comprising epinephrine, levonodefrin, a salt of epinephrine, a salt of levonordefrin, or hydrate thereof.

36. The composition of claim 34, wherein the composition is formulated for injection.

37. The composition of claim 34, wherein the composition is formulated for oral, intraoral, subcutaneous, transdermal, or transmucosal administration.

38. The composition of claim 35, wherein the salt is epinephrine saccharinate, epinephrine acesulfamate, epinephrine glycyrrherinate, epinephrine mono-glycyrrhizinate, epinephrine tri-glycyrrhizinate, epinephrine vanillate, epinephrine ferrulate, epinephrine glycinate, epinephrine cinnamate, epinephrine enoxolone, epinephrine cyclamate, epinephrine steviol, epinephrine aspartamate, epinephrine di-glycyrrhinizinate, epinephrine neotame, levonordefrin saccharinate, levonordefrin acesulfamate, levonordefrin glycyrrherinate, levonordefrin mono-glycyrrhizinate, levonordefrin tri-glycyrrhizinate, levonordefrin vanillate, levonordefrin ferrulate, levonordefrin glycinate, levonordefrin cinnamate, levonordefrin enoxolone, levonordefrin cyclamate, levonordefrin steviol, levonordefrin aspartamate, levonordefrin di-glycyrrhinizinate, or levonordefrin neotame.

39. The compound of claim 1 or hydrate thereof, wherein the pH of the compound is at least 3.0.

40. The composition of claim 34, wherein the pH of the composition ranges from about 3.6 to about 5.5.

41. The composition of claim 34, wherein the composition further comprises at least 0.000001% by weight of epinephrine, levonordefrin, a salt of epinephrine, a salt of levonodefrin, or hydrate thereof.

42. The composition of claim 34, wherein the further composition comprises from about 0.000001% by weight to about 10% by weight of epinephrine, levonordefrin, a salt of epinephrine, a salt of levonodefrin, or hydrate thereof.

43. The composition of claim 34, wherein the composition further comprises about 0.00001% by weight of epinephrine, levonordefrin, a salt of ephineprhine, a salt of levonordefrin, or hydrate thereof.

44. The composition of claim 34, wherein the composition comprises at least 0.0001% by weight of a compound of Formula (I) or hydrate thereof.

45. The composition of claim 34, wherein the composition comprises from about 0.1% by weight to about 10% by weight of a compound of Formula (I) or hydrate thereof.

46. The composition of claim 34, wherein the composition, when formulated for injection, comprises from about 1% by weight to about 3% by weight of a compound of Formula (I) or hydrate thereof.

47. The composition of claim 34, wherein the composition, when formulated for injection, comprises about 1% by weight of a compound of Formula (I) or hydrate thereof.

48. The composition of claim 34, wherein the composition, when formulated for topical or transdermal administration, comprises from about 1% by weight to about 10% by weight of a compound of Formula (I) or hydrate thereof.

49. The composition of claim 34, wherein the composition, when formulated for topical or transdermal administration, comprises about 5% by weight of a compound of Formula (I) or hydrate thereof.

50. A method for making the compound of Formula (I) or hydrate thereof, the method comprising dissolving the compound of Formula (II): 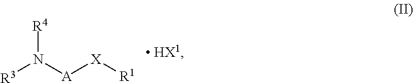
wherein:
X¹ is —OH or a halide anion;
X is —C(O)O—, —OC(O)—, —C(O)NR^{A}—, or —NR^{A}C(O)—, or —CH(OR^{A})—, wherein R^{A} is hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};
R¹ is hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};
each of R³ and R⁴ is independently hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, and heteroaryl is independently substituted with 0-5 occurrences of R^{Z};
A is: 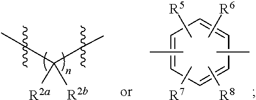
wherein:
n is 1-5;
each of R^{2a} and R^{2b} is independently hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}, or
if n is 1, R^{2a} or R^{2b} and R³ or R⁴, together with the atoms to which they are attached form a 3-8 membered ring independently substituted with 0-5 occurrences of R^{Z}; and
each of R⁵, R⁶, R⁷, and R⁸ is independently hydrogen, alkyl, C₁-C₆ alkoxy, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; and
R^{Z} is halogen, C₁-C₆ alkyl, C₁-C₆ haloalkyl, C₁-C₆ heteroalkyl, —NHR^{Z1}, —NR^{Z1}R^{Z2}, —C(O)R^{Z1}, —C(O)R^{Z2}, —C(O)NR^{Z1}R^{Z2}, —NR^{Z1}C(O)R^{Z2}, —OR^{Z1}, —OR^{Z2}, cyano, or nitro, wherein R^{Z1} is hydrogen, C₁-C₆ alkyl, C₁-C₆ haloalkyl, C₁-C₆ heteroalkyl, hydroxyl, cyano, or nitro and R^{Z2} is carbocyclylalkyl, heterocycylalkyl, aralkyl or heteroaralkyl substituted with 0-5 occurrences of R^{Z1};
provided that the following compounds or hydrates thereof are excluded: 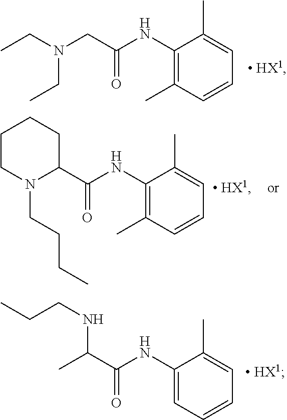
and an organic salt in a solvent, wherein a metal halide precipitates or is solubilized and the compound of Formula (I) or hydrate thereof remains solubilized.

51. The method of claim 50, wherein the organic salt is sodium saccharinate, sodium acesulfamate, sodium glycyrrherinate, sodium mono-glycyrrhizinate, sodium tri-glycyrrhizinate, sodium vanillate, sodium ferrulate, sodium glycinate, sodium cinnamate, sodium enoxolone, sodium cyclamate, sodium steviol, sodium aspartamate, sodium di-glycyrrhinizinate, sodium neotame, potassium saccharinate, potassium acesulfamate, potassium glycyrrherinate, potassium mono-glycyrrhizinate, potassium tri-glycyrrhizinate, potassium vanillate, potassium ferrulate, potassium glycinate, potassium cinnamate, potassium enoxolone, potassium cyclamate, potassium steviol, potassium aspartamate, potassium di-glycyrrhinizinate, or potassium neotame.

52. The method of claim 50, wherein X¹ is chloride or —OH.

53. The method of claim 50, wherein the solvent is acetonitrile.

54. A method of suppressing pain experienced by a subject during a cosmetic, medical, or dental procedure, comprising administering to the subject an effective amount of a composition comprising a compound of Formula (I) or hydrate thereof: 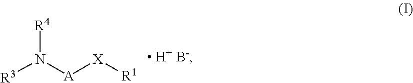
or hydrate thereof, wherein:
B is a sweetener;
X is —C(O)O—, —OC(O)—, —C(O)NR^{A}—, or —NR^{A}C(O)—, or —CH(OR^{A})—, wherein R^{A} is hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};
R¹ is hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z};
each of R³ and R⁴ is independently hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, and heteroaryl is independently substituted with 0-5 occurrences of R^{Z};
A is: 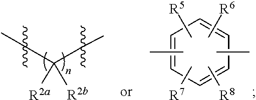
wherein:
n is 1-5;
each of R^{2a} and R^{2b} is independently hydrogen, alkyl, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}, or
if n is 1, R^{2a} or R^{2b} and R³ or R⁴, together with the atoms to which they are attached form a 3-8 membered ring independently substituted with 0-5 occurrences of R^{Z}; and
each of R⁵, R⁶, R⁷, and R⁸ is independently hydrogen, alkyl, C₁-C₆ alkoxy, carbocyclyl, heterocyclyl, carbocyclylalkyl, heterocycylalkyl, aryl, aralkyl, heteroaralkyl, or heteroaryl, wherein each of alkyl, carbocyclyl, heterocyclyl, carbocyclylalkyl, heterocycylalkyl, aryl, aralkyl, heteroaralkyl, and heteroaryl is independently substituted with 0-5 occurrences of R^{Z}; and
R^{Z} is halogen, C₁-C₆ alkyl, C₁-C₆ haloalkyl, C₁-C₆ heteroalkyl, —NHR^{Z1}, —NR^{Z1}R^{Z2}, —C(O)R^{Z1}, —C(O)R^{Z2}, —C(O)NR^{Z1}R^{Z2}, —NR^{Z1}C(O)R^{Z2}, —OR^{Z1}, —OR^{Z2}, cyano, or nitro, wherein R^{Z1} is hydrogen, C₁-C₆ alkyl, C₁-C₆ haloalkyl, C₁-C₆ heteroalkyl, hydroxyl, cyano, or nitro and R^{Z2} is carbocyclylalkyl, heterocycylalkyl, aralkyl or heteroaralkyl substituted with 0-5 occurrences of R^{Z1}.

55. The method of claim 54, wherein the composition comprises lidocaine saccharinate, lidocaine acesulfamate, lidocaine glycyrrherinate, lidocaine mono-glycyrrhizinate, lidocaine tri-glycyrrhizinate, lidocaine vanillate, lidocaine ferrulate, lidocaine glycinate, lidocaine cinnamate, lidocaine enoxolone, lidocaine cyclamate, lidocaine steviol, lidocaine aspartamate, lidocaine di-glycyrrhinizinate, lidocaine neotame, tetracaine saccharinate, tetracaine acesulfamate, tetracaine glycyrrherinate, tetracaine mono-glycyrrhizinate, tetracaine tri-glycyrrhizinate, tetracaine vanillate, tetracaine ferrulate, tetracaine glycinate, tetracaine cinnamate, tetracaine enoxolone, tetracaine cyclamate, tetracaine steviol, tetracaine aspartamate, tetracaine di-glycyrrhinizinate, tetracaine neotame, bupivacaine saccharinate, bupivacaine acesulfamate, bupivacaine glycyrrherinate, bupivacaine mono-glycyrrhizinate, bupivacaine tri-glycyrrhizinate, bupivacaine vanillate, bupivacaine ferrulate, bupivacaine glycinate, bupivacaine cinnamate, bupivacaine enoxolone, bupivacaine cyclamate, bupivacaine steviol, bupivacaine aspartamate, bupivacaine di-glycyrrhinizinate, bupivacaine neotame, mepivacaine saccharinate, mepivacaine acesulfamate, mepivacaine glycyrrherinate, mepivacaine mono-glycyrrhizinate, mepivacaine tri-glycyrrhizinate, mepivacaine vanillate, mepivacaine ferrulate, mepivacaine glycinate, mepivacaine cinnamate, mepivacaine enoxolone, mepivacaine cyclamate, mepivacaine steviol, mepivacaine aspartamate, mepivacaine di-glycyrrhinizinate, mepivacaine neotame, articaine saccharinate, articaine acesulfamate, articaine glycyrrherinate, articaine mono-glycyrrhizinate, articaine tri-glycyrrhizinate, articaine vanillate, articaine ferrulate, articaine glycinate, articaine cinnamate, articaine enoxolone, articaine cyclamate, articaine steviol, articaine aspartamate, articaine di-glycyrrhinizinate, articaine neotame, prilocaine saccharinate, prilocaine acesulfamate, prilocaine glycyrrherinate, prilocaine mono-glycyrrhizinate, prilocaine tri-glycyrrhizinate, prilocaine vanillate, prilocaine ferrulate, prilocaine glycinate, prilocaine cinnamate, prilocaine enoxolone, prilocaine cyclamate, prilocaine steviol, prilocaine aspartamate, prilocaine di-glycyrrhinizinate, prilocaine neotame, procaine saccharinate, procaine acesulfamate, procaine glycyrrherinate, procaine mono-glycyrrhizinate, procaine tri-glycyrrhizinate, procaine vanillate, procaine ferrulate, procaine glycinate, procaine cinnamate, procaine enoxolone, procaine cyclamate, procaine steviol, procaine aspartamate, procaine di-glycyrrhinizinate, procaine neotame, oxybuprocaine saccharinate, oxybuprocaine acesulfamate, oxybuprocaine glycyrrherinate, oxybuprocaine mono-glycyrrhizinate, oxybuprocaine tri-glycyrrhizinate, oxybuprocaine vanillate, oxybuprocaine ferrulate, oxybuprocaine glycinate, oxybuprocaine cinnamate, oxybuprocaine enoxolone, oxybuprocaine cyclamate, oxybuprocaine steviol, oxybuprocaine aspartamate, oxybuprocaine di-glycyrrhinizinate, oxybuprocaine neotame, ropivacaine saccharinate, ropivacaine acesulfamate, ropivacaine glycyrrherinate, ropivacaine mono-glycyrrhizinate, ropivacaine tri-glycyrrhizinate, ropivacaine vanillate, ropivacaine ferrulate, ropivacaine glycinate, ropivacaine cinnamate, ropivacaine enoxolone, ropivacaine cyclamate, ropivacaine steviol, ropivacaine aspartamate, ropivacaine di-glycyrrhinizinate, ropivacaine neotame, cinchocaine saccharinate, cinchocaine acesulfamate, cinchocaine glycyrrherinate, cinchocaine mono-glycyrrhizinate, cinchocaine tri-glycyrrhizinate, cinchocaine vanillate, cinchocaine ferrulate, cinchocaine glycinate, cinchocaine cinnamate, cinchocaine enoxolone, cinchocaine cyclamate, cinchocaine steviol, cinchocaine aspartamate, cinchocaine di-glycyrrhinizinate, cinchocaine neotame, benzocaine saccharinate, benzocaine acesulfamate, benzocaine glycyrrherinate, benzocaine mono-glycyrrhizinate, benzocaine tri-glycyrrhizinate, benzocaine vanillate, benzocaine ferrulate, benzocaine glycinate, benzocaine cinnamate, benzocaine enoxolone, benzocaine cyclamate, benzocaine steviol, benzocaine aspartamate, benzocaine di-glycyrrhinizinate, benzocaine neotame, oxetacaine saccharinate, oxetacaine acesulfamate, oxetacaine glycyrrherinate, oxetacaine mono-glycyrrhizinate, oxetacaine tri-glycyrrhizinate, oxetacaine vanillate, oxetacaine ferrulate, oxetacaine glycinate, oxetacaine cinnamate, oxetacaine enoxolone, oxetacaine cyclamate, oxetacaine steviol, oxetacaine aspartamate, oxetacaine di-glycyrrhinizinate, or oxetacaine neotame.

56. The method of claim 54, wherein the composition further comprises epinephrine, levonordefrin, a salt of epinephrine, a salt of levonodefrin, or hydrate thereof and a pharmaceutically acceptable carrier.

57. The method of claim 56, wherein the salt of epinephrine is epinephrine saccharinate, epinephrine acesulfamate, epinephrine glycyrrherinate, epinephrine mono-glycyrrhizinate, epinephrine tri-glycyrrhizinate, epinephrine vanillate, epinephrine ferrulate, epinephrine glycinate, epinephrine cinnamate, epinephrine enoxolone, epinephrine cyclamate, epinephrine steviol, epinephrine aspartamate, epinephrine di-glycyrrhinizinate, or epinephrine neotame.

58. The method of claim 56, wherein the salt of levonordefrin is levonordefrin saccharinate, levonordefrin acesulfamate, levonordefrin glycyrrherinate, levonordefrin mono-glycyrrhizinate, levonordefrin tri-glycyrrhizinate, levonordefrin vanillate, levonordefrin ferrulate, levonordefrin glycinate, levonordefrin cinnamate, levonordefrin enoxolone, levonordefrin cyclamate, levonordefrin steviol, levonordefrin aspartamate, levonordefrin di-glycyrrhinizinate, or levonordefrin neotame.

59. The method of claim 54, wherein the composition is administered intraorally, epidurally, ocularly, intranasally, transdermally, subcutaneously, intramuscularly, or transmucosally.

60. The method of claim 54, wherein the composition is formulated for injection.

61. The method of claim 54, wherein the pH of the compound of Formula (I) or hydrate thereof is at least 3.0.

62. The method of claim 54, wherein the pH of the compound of Formula (I) or hydrate thereof ranges from about 3.5 to about 5.5.

63. The method of claim 54, wherein the pH of a composition comprising a compound of Formula (I) or hydrate thereof is at least 3.0.

64. The method of claim 54, wherein the pH of a composition comprising a compound of Formula (I) or hydrate thereof ranges from about 3.6 to about 5.5.

65. The method of claim 54, wherein the composition further comprises at least 0.000001% by weight of epinephrine, levonordefrin, a salt of epinephrine, a salt of levonodefrin, or hydrate thereof.

66. The method of claim 54, wherein the composition further comprises from about 0.000001% by weight to about 10% by weight of epinephrine, levonordefrin, a salt of epinephrine, a salt of levonodefrin, or hydrate thereof.

67. The method of claim 54, wherein the composition further comprises about 0.00001% by weight of epinephrine, levonordefrin, a salt of epinephrine, a salt of levonodefrin, or hydrate thereof.

68. The method of claim 54, wherein the composition further comprises at least 0.0001% by weight of a compound of Formula (I) or hydrate thereof.

69. The method of claim 54, wherein the composition further comprises from about 0.01% by weight to about 10% by weight of a compound of Formula (I) or hydrate thereof.

70. The method of claim 54, wherein the composition, when formulated for injection, comprises from about 1% by weight to about 3% by weight of a compound of Formula (I) or hydrate thereof.

71. The method of claim 54, wherein the composition, when formulated for injection, comprises about 1% by weight of a compound of Formula (I) or hydrate thereof.

72. The method of claim 54, wherein the composition, when formulated for topical or transdermal administration, comprises from about 1% by weight to about 10% by weight of a compound of Formula (I) or hydrate thereof.

73. The method of claim 54, wherein the composition, when formulated for topical or transdermal administration, comprises about 5% by weight of a compound of Formula (I) or hydrate thereof.
